(19) United States
(12) Patent Application Publication
(10) Pub. No.: US 2024/0165247 A1
(43) Pub. Date: May 23, 2024

(54) NEW PEPTIDE CONJUGATES

(71) Applicant: JIANGYIN USUN PHARMACEUTICAL CO., LTD., Jiangyin, Jiangsu (CN)

(72) Inventors: Ming GU, Jiangyin, Jiangsu (CN); Maoqian SONG, Jiangyin, Jiangsu (CN); Bengt Ingemar SAMUELSSON, Solna (SE); Jan-Christer JANSON, Uppsala (SE)

(21) Appl. No.: 18/281,859

(22) PCT Filed: Mar. 17, 2021
(86) PCT No.: PCT/CN2021/081361

§ 371 Date: Sep. 13, 2023

(51) Int. Cl. A61K 47/64; A61K 6/69; A61P 29/00; C07K 7/06

(52) U.S. Cl. CPC A61K 47/64; A61K 6/69; A61P 29/00; C07K 7/06

(57) ABSTRACT

Provided are a conjugate formed between an anaesthetic compound and a peptide component of the amino acid sequence: W-Lys-X1-Ser-U-X2-Y, wherein: W, X1, U, X2 and Y are as defined in the description, as well as regioisomers, stereoisomers, and pharmaceutically- or cosmetically-acceptable salts of said conjugates, which conjugates are useful in the treatment of conditions characterised by inflammation and/or of pain. Preferred anaesthetics are local anaesthetics, such as procaine.

### FIELD OF THE INVENTION

This invention relates to new peptide conjugate compounds, the use of such conjugates in human medicine, and to pharmaceutical compositions comprising them. In particular, the invention relates to the use of those conjugates and compositions in the treatment of e.g. inflammation and/or pain.

### Background and Prior Art

Inflammation is typically characterized as a localised tissue response to e.g. invasion of microorganisms, certain antigens, damaged cells or physical and/or chemical factors. The inflammatory response is normally a protective mechanism which serves to destroy, dilute or sequester both the injurious agent and the injured tissue, as well as to initiate tissue healing.

Inflammation may result from physical trauma, infection, some chronic diseases (e.g. psoriasis and autoimmune diseases, such as rheumatoid arthritis) and/or chemical and/or physiological reactions to external stimuli (e.g. as part of an allergic response). A complex series of events may be involved, in which inflammatory mediators increase blood flow and dilation of local blood vessels, resulting in redness and heat, the exudation of fluids, often resulting in localised swelling, leukocytic migration into the inflamed area, and pain.

Many conditions/disorders are characterized by, and/or are caused by, abnormal, tissue-damaging inflammation. Such conditions are typically characterized by activation of immune defence mechanisms, resulting in an effect that is more harmful than beneficial to the host, and are generally associated with varying degrees of tissue redness or hyperemia, swelling, hyperthermia, pain, itching, cell death, tissue destruction, cell proliferation and/or loss of function. Examples include inflammatory bowel diseases, rheumatoid arthritis, multiple sclerosis, psoriasis, glomerulonephritis and transplant rejection.

Typically, a complex series of events results in inflammatory changes such as increased blood flow through dilation of local blood vessels, resulting in redness and heat, the extravasation of leukocytes and plasma, often resulting in localised swelling, activation of sensory nerves (resulting in pain in some tissues) and loss of function. These inflammatory changes are triggered by a cascade of cellular and biochemical events involving cells like neutrophils, monocytes, macrophages and lymphocytes together with inflammatory mediators such as vasoactive amines, cytokines, complement factors and reactive oxygen species.

Amongst other things, inflammation plays a key role in the wound healing process. Wounds and burns can therefore be classified as conditions with which inflammation is associated. Traditional thinking in the art is that anti-inflammatory drugs should not be applied directly to open wounds, as this would be detrimental to the progress of wound healing.

Fibrosis is defined by the excessive accumulation of fibrous connective tissue (components of the extracellular matrix (ECM) such as collagen and fibronectin) in and around inflamed or damaged tissue. Although collagen deposition is typically a reversible part of wound healing, it can often evolve into a progressively irreversible fibrotic response if tissue injury is severe, or if the wound-healing response itself becomes dysregulated. Furthermore, fibrogenesis is known to be a major cause of morbidity and mortality in many chronic inflammatory diseases, as well as end-stage liver disease, kidney disease, idiopathic pulmonary fibrosis (IPF) and heart failure. It is also a pathological feature of many chronic autoimmune diseases, such as scleroderma, rheumatoid arthritis, Crohn's disease, ulcerative colitis, myelofibrosis and systemic lupus erythematosus. Fibrosis may also influence the pathogenesis of many progressive myopathies, metastasis and graft rejection.

Irrespective of whether it is caused by, and/or it is associated with, inflammation, pain control is of prime importance in the treatment of many different diseases and medical conditions. Proper pain relief imparts significant physiological and psychological benefits to the patient. Not only does effective pain relief mean a smoother, more pleasant recovery (e.g., mood, sleep, quality of life, etc.) with earlier discharge from medical/surgical/outpatient facilities, but it may also reduce the probability of the acute pain state progressing to a chronic pain syndrome.

Mussel adhesive protein (MAP), also known as Mytilus edulis foot protein (mefp), is a protein that is secreted by marine shellfish species, such as Mytilus edulis, Mytilus coruscus and Perna viridis. Eleven identified separate adhesive protein subtypes have been derived from mussels, including the collagens pre-COL-P, pre-COL-D and pre-COL-NG; the mussel feet matrix proteins PTMP (proximal thread matrix protein) and DTMP (distal thread matrix protein); and mfp proteins mfp-2 (sometimes referred to as “mefp-2”, hereinafter used interchangeably), mfp-3/mefp-3, mfp-4/mefp-4, mfp-5/mefp-5, mfp-6/mefp-6 and, most preferably mfp-1/mefp-1 (see, for example, Zhu et al., Advances in Marine Science, 2014, 32, 560-568 and Gao et al., Journal of Anhui Agr. Sci., 2011, 39, 19860-19862).

A significant portion of mefp-1 consists of 70 to 90 tandem repeats of the decapeptide: Ala-Lys-Pro-Ser-Tyr-Hyp-Hyp-Thr-DOPA-Lys (SEQ ID No: 1; see Waite, Int. J. Adhesion and Adhesives, 1987, 7, 9-14). This decapeptide sequence may be isolated as a low molecular weight derivative of naturally-occurring MAPs, or may be synthesized, for example as described by Yamamoto in J. Chem. Soc., Perkin Trans., 1987, 1, 613-618. See also Dalsin et al., J. Am. Chem. Soc., 2003, 125, 4253-4258.

Analogues of the decapeptide, notably Ala-Lys-Pro-Ser-Tyr-Hyp-Hyp-Thr-Tyr-Lys (SEQ ID No: 2) have also been disclosed. See, for example, U.S. Pat. No. 5,616,311 and WO 96/39128.

Anaesthetics (both local and general) are frequently employed to treat acute and chronic pain. Such pain may be associated with disorders (characterised by or associated with inflammation or otherwise), wounds or burns. Anaesthetics are also routinely employed in advance or, during or after surgical and/or diagnostic interventions carried out on the human body.

Local anaesthetics act by binding to fast sodium channels from within (in an open state), and so prevent transmission of nerve impulses without causing unconsciousness. They can be either ester- or amide-based. General anaesthetics tend to cause sedation and therefore may act by one or more of numerous mechanisms.

There is a clear need for new and/or improved medicines that may be used in the treatment of inflammation, conditions characterised thereby, and/or in the treatment of pain.

### DISCLOSURE OF THE INVENTION

According to a first aspect of the invention, there is provided a conjugate compound formed between an anaesthetic compound and a peptide component, preferably selected from the amino acid sequence:

W-Lys-X¹-Ser-U-X²-Y (SEQ ID No: 3)
wherein:
W is absent (in which case Lys is the N-terminal amino acid), or represents a 1, 2 or 3 amino acid sequence, in which the amino acids are selected from one or more of the group Ser, Lys, Ala, DOPA and a 3,4-dihydrocinnamic acid (HCA) residue, provided that, when present, the HCA residue is located at the N-terminus of the peptide sequence;
X¹ represents Pro, Hyp or diHyp;
U represents Tyr, DOPA or a single bond (i.e. is absent);
X² represents Ser, Pro, Hyp or diHyp; and
Y represents a 1 to 5 (e.g. a 1 to 4) amino acid sequence, in which the amino acids are selected from one or more of the group Lys, Ala, Pro, Hyp, diHyp, Thr, DOPA and Tyr, as well as regioisomers, stereoisomers, and pharmaceutically- or and/or cosmetically-acceptable salts of said conjugates, which conjugate compounds, regioisomers, stereoisomers and salts are referred to together hereinafter as ‘the conjugates of the invention’.

The ‘anaesthetic compound’ includes any compound that is capable of inducing anaesthesia at a local and/or systemic level with a view to avoiding severe pain for a multitude of reasons, including those described hereinafter. Anaesthetic compounds may thus be locally acting (a local anaesthetic) or generally acting (a general anaesthetic),

General anaesthetics are typically administered prior to the occurrence of pain (e.g. prior to surgical or other interventions) and may include any agent that is capable of providing reduce consciousness (such as unconsciousness) and/or sedation. General anaesthetics thus include inhaled gases, such as desflurane, enflurane, halothane, isoflurane, methoxyflurane, nitrous oxide, sevoflurane, xenon; barbiturates, such as amobarbital, methohexital, thiamylal and thiopental; benzodiazepines, such as diazepam, lorazepam, midazolam, etomidate, ketamine and propofol; short-acting opioids, such as alfentanil, fentanyl, remifentanil and sufentanil.

However, it is preferred that the anaesthetic component of a conjugate of the invention comprises a local anaesthetic.

The term ‘local anaesthetic’ includes any active pharmaceutical compound that causes absence of the sensation of pain in a specific location of the body without a loss of consciousness, is capable of binding to sodium channels, and/or is capable of preventing transmission of nerve impulses. Such locations may be very localised (e.g. intraorally, in the case of a tooth that needs to be removed) or around a wound that requires stitches, or can be regional anesthesia, which is used for larger areas of the body such as an arm, a leg or in obstetrics, etc.

Local anaesthetics may be selected from the group amylocaine, ambucaine, articaine, benzocaine, benzonatate, bupivacaine, butacaine, butanilicaine, chloroprocaine, cinchocaine, cocaine, cyclomethycaine, dibucaine, diperodon, dimethocaine, eucaine, etidocaine, hexylcaine, fomocaine, fotocaine, hydroxyprocaine, isobucaine, levobupivacaine, lidocaine/lignocaine, mepivacaine, meprylcaine, metabutoxycaine, nitracaine, orthocaine, oxetacaine (oxethazaine), oxybuprocaine, paraethoxycaine, phenacaine, piperocaine, piridocaine, pramocaine, prilocaine, primacaine, procaine, procainamide, propantheline, proparacaine, propoxycaine, pyrrocaine, quinisocaine, risocaine, ropivacaine, trimecaine, tetracaine, tolycaine and tropacocaine.

Preferred local anaesthetics include those that contain one or more free amino groups, and may thus be selected from the group ambucaine, benzocaine, butacaine, chloroprocaine, dimethocaine, metabutoxycaine, orthocaine, propantheline, propoxycaine, risocaine and, particularly, procaine.

Peptide components that may be mentioned include those in which:
W represents a 1 or 2 amino acid sequence, in which the amino acids are selected from one or more of the group HCA and, more preferably, Lys, Ala and DOPA;
U represents Tyr or DOPA;
Y represents a 1 to 5 (e.g. a 1 to 4) amino acid sequence, in which the amino acids are selected from one or more of the group Lys, Ala, Pro, Hyp, Thr, DOPA and Tyr.

Preferred conjugates of the invention include those in which:
X¹ represents Hyp or, more preferably, Pro;
X² represents Ser, Pro or, more preferably, Hyp;
W represents HCA, HCA-Ala-, preferably Ala or Lys-Ala or, more preferably DOPA or DOPA-Ala-; and/or
Y represents a 5, preferably a 3 or, more preferably, a 4 amino acid sequence, in which the amino acids are selected from one or more of the group Lys, Ala, Hyp, Thr, DOPA and Tyr.

More preferably, conjugates of the invention include those in which Y represents a 4 amino acid sequence selected from the group -Pro-Y¹-Y²-Lys- or, more preferably, -Hyp-Y¹-Y²-Lys- and -Thr-Y¹-Y²-Lys-, wherein Y¹ and Y² are each independently selected from the group Pro or, more preferably, Ala, Hyp, Thr, DOPA and Tyr.

Wherein Y represents a 4 amino acid sequence, preferred conjugates of the invention include those in which the amino acid sequence defined by Y is selected from the group:
Pro-Thr-DOPA-Lys-;
Pro-Thr-Tyr-Lys-;
Thr-Tyr-Pro-Lys-;
Thr-DOPA-Pro-Lys-; and, more preferably,
Hyp-Thr-Tyr-Lys-;
Hyp-Thr-DOPA-Lys-;
Hyp-Thr-Ala-Lys-;
Thr-Tyr-Hyp-Lys-;
Thr-DOPA-Hyp-Lys-; and
Thr-Ala-Hyp-Lys-.

When Y represents a 2 amino acid sequence, preferred conjugates of the invention include those in which the amino acid sequence defined by Y is selected from the group -Hyp-Thr-, -Thr-Tyr-, -Pro-Thr- and -Thr-DOPA-.

Other preferred conjugates of the invention that may be mentioned include those in which the amino acid sequence defined by Y is selected from -Thr-Tyr-Lys-, -Tyr-Pro-Lys-, -DOPA-Pro-Lys-, -Hyp-Thr-Tyr-, -Hyp-Thr-Tyr-Hyp-Lys- and, more preferably, the groups -Thr-Tyr-Hyp-Lys-DOPA- and -Hyp-Thr-DOPA-.

Conjugates of the invention that may be mentioned include those in which:
X¹ represents Pro;
U represents Tyr; and/or
represents Ala, and, in this respect, conjugates of the invention that may be mentioned include those of the amino acid sequence:

(SEQ ID No: 4)
Ala-Lys-Pro-Ser-Tyr-Hyp-Thr-Tyr-Hyp-Lys-DOPA;
(SEQ ID No: 5)
Ala-Lys-Pro-Ser-Tyr-Pro-Pro-Thr-DOPA-Lys;
(SEQ ID No: 6)
Ala-Lys-Pro-Ser-Tyr-Pro-Thr-Tyr-Pro-Lys;
(SEQ ID No: 7)
Ala-Lys-Pro-Ser-Tyr-Pro-Thr-DOPA-Pro-Lys;
(SEQ ID No: 8)
Ala-Lys-Pro-Ser-Tyr-Pro-Hyp-Thr-Tyr-Lys;
(SEQ ID No: 9)
Ala-Lys-Pro-Ser-Tyr-Pro-Hyp-Thr-DOPA-Lys;
(SEQ ID No: 10)
Ala-Lys-Pro-Ser-Tyr-Hyp-Pro-Thr-Tyr-Lys;
(SEQ ID No: 11)
Ala-Lys-Pro-Ser-Tyr-Hyp-Pro-Thr-DOPA-Lys;
(SEQ ID No: 12)
Ala-Lys-Pro-Ser-Tyr-Hyp-Thr-Tyr-Hyp-Lys;
(SEQ ID No: 13)
Ala-Lys-Pro-Ser-Tyr-Hyp-Thr-DOPA-Hyp-Lys;
(SEQ ID No: 14)
Ala-Lys-Pro-Ser-Tyr-Hyp-Hyp-Thr-Tyr-Lys-DOPA;
(SEQ ID No: 15)
Ala-Lys-Pro-Ser-Tyr-Hyp-Hyp-Thr-DOPA-Lys-DOPA;
and
(SEQ ID NO: 16)
Ala-Lys-Pro-Ser-Tyr-Hyp-Thr-DOPA-Hyp-Lys-DOPA.

Conjugates of the invention that may be mentioned include those in which:
U represents Tyr;
X² represents Hyp; and/or
W represents Lys-Ala-, and, in this respect, conjugates of the invention that may be mentioned include those of the amino acid sequence:

(SEQ ID No: 17)
Lys-Ala-Lys-Pro-Ser-Tyr-Hyp-Hyp-Thr-DOPA;
(SEQ ID No: 18)
Lys-Ala-Lys-Hyp-Ser-Tyr-Hyp-Hyp-Thr-DOPA;
(SEQ ID No: 19)
Lys-Ala-Lys-Hyp-Ser-Tyr-Hyp-Hyp-Thr-Tyr;
and
(SEQ ID NO: 20)
Lys-Ala-Lys-Pro-Ser-Tyr-Hyp-Hyp-Thr-Tyr.

Further conjugates of the invention that may be mentioned include those in which:
X¹ represents Pro;
U represents Tyr;
X² represents Hyp; and/or
W represents HCA, HCA-Ala- or, more preferably, DOPA or DOPA-Ala-, and, in this respect, conjugates of the invention that may be mentioned include those of the amino acid sequence:

(SEQ ID No: 21)
DOPA-Lys-Pro-Ser-Tyr-Hyp-Thr-Ala-Hyp-Lys;
(SEQ ID No: 22)
DOPA-Lys-Pro-Ser-Tyr-Hyp-Hyp-Thr-Ala-Lys;
(SEQ ID No: 23)
DOPA-Ala-Lys-Pro-Ser-Tyr-Hyp-Thr-Tyr-Hyp-Lys;
(SEQ ID No: 24)
DOPA-Ala-Lys-Pro-Ser-Tyr-Hyp-Hyp-Thr-Tyr-Lys;
(SEQ ID No: 25)
DOPA-Ala-Lys-Pro-Ser-Tyr-Hyp-Thr-DOPA-Hyp-Lys;
(SEQ ID No: 26)
DOPA-Ala-Lys-Pro-Ser-Tyr-Hyp-Hyp-Thr-DOPA-Lys;
(SEQ ID No: 27)
HCA-Lys-Pro-Ser-Tyr-Hyp-Hyp-Thr-Ala-Lys;
(SEQ ID No: 28)
HCA-Ala-Lys-Pro-Ser-Tyr-Hyp-Thr-Tyr-Hyp-Lys;
(SEQ ID No: 29)
HCA-Ala-Lys-Pro-Ser-Tyr-Hyp-Thr-DOPA-Hyp-Lys;
(SEQ ID No: 30)
HCA-Ala-Lys-Pro-Ser-Tyr-Hyp-Hyp-Thr-Tyr-Lys;
(SEQ ID No: 31)
HCA-Ala-Lys-Pro-Ser-Tyr-Hyp-Hyp-Thr-DOPA-Lys;
and
(SEQ ID NO: 32)
HCA-Lys-Pro-Ser-Tyr-Hyp-Thr-Ala-Hyp-Lys.

Other conjugates of the invention that may be mentioned include those in which:
U represents DOPA; and/or
W represents Ala or Lys-Ala-, and, in this respect, conjugates of the invention that may be mentioned include those of the amino acid sequence:

(SEQ ID No: 33)
Lys-Ala-Lys-Hyp-Ser-DOPA-Hyp-Hyp-Thr-DOPA;
(SEQ ID No: 34)
Lys-Ala-Lys-Pro-Ser-DOPA-Hyp-Hyp-Thr-DOPA;
(SEQ ID No: 35)
Ala-Lys-Pro-Ser-DOPA-Pro-Pro-Thr-Tyr-Lys;
(SEQ ID No: 36)
Ala-Lys-Pro-Ser-DOPA-Pro-Pro-Thr-DOPA-Lys;
(SEQ ID No: 37)
Ala-Lys-Pro-Ser-DOPA-Pro-Thr-Tyr-Pro-Lys;
(SEQ ID No: 38)
Ala-Lys-Pro-Ser-DOPA-Pro-Thr-DOPA-Pro-Lys;
(SEQ ID No: 39)
Ala-Lys-Pro-Ser-DOPA-Pro-Hyp-Thr-Tyr-Lys;
(SEQ ID No: 40)
Ala-Lys-Pro-Ser-DOPA-Pro-Hyp-Thr-DOPA-Lys;
(SEQ ID No: 41)
Ala-Lys-Pro-Ser-DOPA-Hyp-Pro-Thr-Tyr-Lys;
(SEQ ID No: 42)
Ala-Lys-Pro-Ser-DOPA-Hyp-Pro-Thr-DOPA-Lys;
(SEQ ID No: 43)
Ala-Lys-Pro-Ser-DOPA-Hyp-Thr-Tyr-Hyp-Lys;
(SEQ ID No: 44)
Ala-Lys-Pro-Ser-DOPA-Hyp-Thr-DOPA-Hyp-Lys;
(SEQ ID No: 45)
Lys-Ala-Lys-Pro-Ser-DOPA-Hyp-Hyp-Thr-Tyr;
(SEQ ID No: 46)
Lys-Ala-Lys-Hyp-Ser-DOPA-Hyp-Hyp-Thr-Tyr;
(SEQ ID No: 47)
Ala-Lys-Pro-Ser-DOPA-Hyp-Hyp-Thr-Tyr-Lys-DOPA;
(SEQ ID No: 48)
Ala-Lys-Pro-Ser-DOPA-Hyp-Hyp-Thr-DOPA-Lys-DOPA;
(SEQ ID No: 49)
Ala-Lys-Pro-Ser-DOPA-Hyp-Thr-Tyr-Hyp-Lys-DOPA;
and
(SEQ ID No: 50)
Ala-Lys-Pro-Ser-DOPA-Hyp-Thr-DOPA-Hyp-Lys-DOPA.

Further conjugates of the invention that may be mentioned include those in which:
X¹ represents Pro;
U represents DOPA;
X² represents Hyp; and/or
W represents HCA, HCA-Ala- or, more preferably, DOPA or DOPA-Ala-, and, in this respect, conjugates of the invention that may be mentioned include those of the amino acid sequence:

(SEQ ID No: 51)
DOPA-Lys-Pro-Ser-DOPA-Hyp-Thr-Ala-Hyp-Lys;
(SEQ ID No: 52)
DOPA-Lys-Pro-Ser-DOPA-Hyp-Hyp-Thr-Ala-Lys;
(SEQ ID No: 53)
DOPA-Ala-Lys-Pro-Ser-DOPA-Hyp-Thr-DOPA-Hyp-Lys;
(SEQ ID No: 54)
DOPA-Ala-Lys-Pro-Ser-DOPA-Hyp-Hyp-Thr-Tyr-Lys;
(SEQ ID No: 55)
DOPA-Ala-Lys-Pro-Ser-DOPA-Hyp-Hyp-Thr-DOPA-Lys.
(SEQ ID No: 56)
HCA-Ala-Lys-Pro-Ser-DOPA-Hyp-Thr-DOPA-Hyp-Lys;
(SEQ ID No: 57)
HCA-Ala-Lys-Pro-Ser-DOPA-Hyp-Hyp-Thr-DOPA-Lys;
(SEQ ID No: 58)
HCA-Lys-Pro-Ser-DOPA-Hyp-Thr-Ala-Hyp-Lys;
(SEQ ID No: 59)
HCA-Lys-Pro-Ser-DOPA-Hyp-Hyp-Thr-Ala-Lys;
(SEQ ID No: 60)
DOPA-Ala-Lys-Pro-Ser-DOPA-Hyp-Thr-Tyr-Hyp-Lys;
(SEQ ID No: 61)
HCA-Ala-Lys-Pro-Ser-DOPA-Hyp-Thr-Tyr-Hyp-Lys;
and
(SEQ ID NO: 62)
HCA-Ala-Lys-Pro-Ser-DOPA-Hyp-Hyp-Thr-Tyr-Lys.

Peptide components that may be included in conjugates of the invention that may be mentioned include those of the amino acid sequence:

(SEQ ID NO: 63)
K-W¹-Lys-X¹-Ser-U-X2-Y¹-I-J

wherein K represents an optional N-terminal HCA group;
W¹ may be absent (in which case Lys is the N-terminal amino acid) or W¹ may represent a 1 or 2 amino acid sequence, in which the amino acids are selected from one or more of the group Ser, Lys, Ala and DOPA;
Y¹ represents a single bond or a 1 to 3 (e.g. a 1 or 2) amino acid sequence, in which the amino acids are selected from one or more of the group Lys, Ala, Pro, Hyp, diHyp, Thr, DOPA and Tyr;
I represents Pro, Hyp, diHyp, Thr, DOPA or Tyr;
J represents Lys or is absent (in which case I represents the C-terminal amino acid); and
X¹ and X² are as hereinbefore defined.

When conjugates of the invention comprise a peptide component of SEQ ID No: 63, those that may be mentioned include those in which:
W¹ represents Ala or Ser, or is absent (in which case, Lys is the N-terminal amino acid);
X² represents Pro, Hyp or diHyp; and/or
when K is not present, W¹ represents Ala or is absent and J represents Lys, then I represents Pro, Hyp, diHyp or Thr (i.e. I does not represent DOPA or Tyr).

Preferred conjugates of the invention comprising a peptide component of SEQ ID No: 63 include those in which:
U represents DOPA or, more preferably Tyr;
X¹ represents Hyp or, more preferably, Pro;
X² represents diHyp or, more preferably, Hyp; and/or
Y¹ represents a 3, a 1 or, preferably, a 2 amino acid sequence, in which the amino acids are selected from the group Pro, Hyp, Thr, DOPA and Tyr.

Peptide components of SEQ ID No: 63 that may be mentioned include those in which W¹ represents Ser.

However, more preferred peptide components of SEQ ID No: 63 include those in which W¹ is absent or, more preferably, W¹ represents Ala.

Preferred peptide components of SEQ ID No: 63 also include those in which J represents Lys.

More preferably, peptide components of SEQ ID No: 63 also include those in which I represents DOPA or Tyr, more preferably Pro or, especially, Hyp.

Preferred peptide components of SEQ ID No: 63 also include those in which, when J represents Lys, I represents DOPA or Tyr, more preferably Pro or, especially, Hyp.

Preferred peptide components of SEQ ID No: 63 also include those in which J is absent.

Preferred peptide components of SEQ ID No: 63 also include those in which, when J is absent, I represents DOPA or Tyr, more preferably Pro or, especially Hyp.

Further preferred peptide components of SEQ ID No: 63 include those in which the amino acids in the sequence defined by Y¹ are selected from Pro, preferably DOPA, more preferably Hyp, Thr and Tyr.

Especially preferred peptide components of SEQ ID No: 63 include those in which, in the sequence defined by Y¹:
the amino acid DOPA, preferably Thr or Lys or, more preferably, Tyr is linked to I; and/or the amino acid Pro, or more preferably Hyp or Thr is linked to X².

Preferred values of Y¹ in peptide components of SEQ ID No: 63 above include, when it is a 3-membered amino acid sequence, -Hyp-Thr-Tyr- or, more preferably -Hyp-Thr-DOPA-, -Thr-DOPA-Lys or -Thr-Tyr-Lys-, and, when it is a 2-membered amino acid sequence, -Thr-Tyr- or, more preferably, -Thr-DOPA-, -Pro-Thr- or, more preferably, -Hyp-Thr-.

Particular conjugates of the invention comprising peptide components of SEQ ID No: 63 that may be mentioned include those in which K is absent.

In this respect, peptide components of SEQ ID No: 63 include those comprising the amino acid sequence:

(SEQ ID No: 64)
Lys-Pro-Ser-Tyr-Hyp-Hyp-Thr-DOPA-Hyp-Lys;
(SEQ ID No: 65)
Lys-Pro-Ser-Tyr-Hyp-Hyp-Thr-Tyr-Hyp-Lys;
(SEQ ID No: 66)
Lys-Pro-Ser-Tyr-Hyp-Thr-DOPA-Hyp-Lys;
(SEQ ID No: 67)
Lys-Pro-Ser-Tyr-Hyp-Thr-Tyr-Hyp-Lys;
(SEQ ID No: 68)
Ser-Lys-Pro-Ser-Tyr-Hyp-Hyp-Thr-DOPA-Lys;
(SEQ ID No: 69)
Ser-Lys-Pro-Ser-Tyr-Hyp-Hyp-Thr-Tyr-Lys;
(SEQ ID No: 70)
Ser-Lys-Pro-Ser-Tyr-Hyp-Thr-DOPA-Hyp-Lys;
(SEQ ID NO: 71)
Ser-Lys-Pro-Ser-Tyr-Hyp-Thr-Tyr-Hyp-Lys.

More preferred conjugates of the invention comprising peptide components of SEQ ID No: 63 include those comprising the amino acid sequence:

(SEQ ID No: 72)
Ala-Lys-Pro-Ser-Tyr-Hyp-Hyp-Thr-DOPA-Hyp-Lys;
(SEQ ID No: 73)
Ala-Lys-Pro-Ser-Tyr-Hyp-Hyp-Thr-Tyr-Hyp-Lys;

more preferably those comprising the amino acid sequence:

(SEQ ID No: 74)
Ala-Lys-Pro-Ser-Tyr-Hyp-Thr-DOPA-Hyp-Lys;

and

particularly those comprising the amino acid sequence:

(SEQ ID NO: 75)
Ala-Lys-Pro-Ser-Tyr-Hyp-Thr-Tyr-Hyp-Lys.

Further conjugates of the invention comprising peptide components of SEQ ID No: 63 that may be mentioned include those in which J is absent, such as those comprising the amino acid sequence:

(SEQ ID No: 76)
Lys-Pro-Ser-Tyr-Hyp-Hyp-Thr-DOPA-Hyp;
(SEQ ID No: 77)
Lys-Pro-Ser-Tyr-Hyp-Hyp-Thr-Tyr-Hyp;
(SEQ ID No: 78)
Lys-Pro-Ser-Tyr-Hyp-Thr-DOPA-Hyp;
(SEQ ID No: 79)
Lys-Pro-Ser-Tyr-Hyp-Thr-Tyr-Hyp;
(SEQ ID No: 80)
Ser-Lys-Pro-Ser-Tyr-Hyp-Hyp-Thr-Tyr;
(SEQ ID No: 81)
Ser-Lys-Pro-Ser-Tyr-Hyp-Thr-DOPA-Hyp;
(SEQ ID No: 82)
Ser-Lys-Pro-Ser-Tyr-Hyp-Thr-Tyr-Hyp;
(SEQ ID No: 83)
Ala-Lys-Pro-Ser-Tyr-Hyp-Hyp-Thr-DOPA-Hyp;
(SEQ ID No: 84)
Ala-Lys-Pro-Ser-Tyr-Hyp-Hyp-Thr-Tyr-Hyp;
(SEQ ID No: 85)
Ala-Lys-Pro-Ser-Tyr-Hyp-Thr-DOPA-Hyp;
(SEQ ID No: 86)
Ala-Lys-Pro-Ser-Tyr-Hyp-Thr-Tyr-Hyp;
and
particularly, those comprising
the amino acid sequence:
(SEQ ID NO: 87)
Ser-Lys-Pro-Ser-Tyr-Hyp-Hyp-Thr-DOPA.

Further conjugates of the invention comprising peptide components of SEQ ID No: 63 include those in which K is an N-terminal HCA group, include those comprising the amino acid sequence:

(SEQ ID No: 88)
HCA-Ala-Lys-Pro-Ser-Tyr-Hyp-Hyp-Thr-Tyr-Lys;

and, more preferably, that defined by the amino acid sequence:

(SEQ ID NO: 89)
HCA-Ala-Lys-Pro-Ser-Tyr-Hyp-Thr-Tyr-Hyp-Lys.

Further preferred conjugates of the invention comprising peptide components of SEQ ID No: 63 that may be mentioned include those in which W¹ is Ala and J is Lys, such as those comprising the amino acid sequence:

(SEQ ID No: 90)
Ala-Lys-Pro-Ser-DOPA-Hyp-Thr-DOPA-Hyp-Lys;
(SEQ ID No: 91)
Ala-Lys-Pro-Ser-Pro-Thr-Tyr-Pro-Lys;
(SEQ ID No: 92)
Ala-Lys-Hyp-Ser-Tyr-Hyp-Thr-Tyr-Hyp-Lys;
(SEQ ID No: 93)
Ala-Lys-Hyp-Ser-DOPA-Hyp -Thr-DOPA-Hyp-Lys;

and particularly, those defined by the amino acid sequence:

(SEQ ID NO: 94)
Ala-Lys-Hyp-Ser-Tyr-Hyp-Thr-DOPA-Hyp-Lys.

Further preferred conjugates of the invention comprising peptide components of SEQ ID No: 63 that may be mentioned include those in which J is absent, such as those comprising the amino acid sequence:

(SEQ ID No: 95)
HCA-Ala-Lys-Pro-Ser-Tyr-Hyp-Hyp-Thr-Tyr;
(SEQ ID No: 96)
HCA-Ala-Lys-Pro-Ser-Tyr-Hyp-Thr-Tyr-Hyp;
(SEQ ID No: 97)
Ala-Lys-Pro-Ser-DOPA-Hyp-Thr-DOPA-Hyp;
(SEQ ID No: 98)
Ala-Lys-Pro-Ser-Pro-Thr-Tyr-Pro;
(SEQ ID No: 99)
Ala-Lys-Hyp-Ser-Tyr-Hyp-Thr-Tyr-Hyp;
(SEQ ID No: 100)
Ala-Lys-Hyp-Ser-DOPA-Hyp -Thr-DOPA-Hyp;
(SEQ ID No: 101)
Ala-Lys-Hyp-Ser-Tyr-Hyp-Thr-DOPA-Hyp;
(SEQ ID No: 102)
Ala-Lys-Pro-Ser-DOPA-Hyp-Thr-Tyr-Lys-Hyp;
(SEQ ID No: 103)
Ala-Lys-Hyp-Ser-DOPA-Hyp-Thr-Tyr-Lys-Hyp;
(SEQ ID No: 104)
Ala-Lys-Pro-Ser-Tyr-Hyp-Thr-Tyr-Lys-Hyp;
(SEQ ID No: 105)
Ala-Lys-Hyp-Ser-Tyr-Hyp-Thr-Tyr-Lys-Hyp;
(SEQ ID No: 106)
Ala-Lys-Pro-Ser-Tyr-Hyp-Thr-DOPA-Lys-Hyp;
(SEQ ID No: 107)
Ala-Lys-Hyp-Ser-Tyr-Hyp-Thr-DOPA-Lys-Hyp;
(SEQ ID No: 108)
Ala-Lys-Pro-Ser-DOPA-Hyp-Thr-DOPA-Lys-Hyp;
and
(SEQ ID NO: 109)
Ala-Lys-Hyp-Ser-DOPA-Hyp-Thr-DOPA-Lys-Hyp.

Other conjugates of the invention comprising peptide components of SEQ ID No: 63 that may be mentioned include those in which K and W¹ are both absent and Y¹ represents a single bond.

More preferred conjugates of the invention comprising peptide components of SEQ ID No: 63, in which K and W 1 are both absent and Y¹ represents a single bond, include, in particular, those in which J represents Lys. Such peptide components are necessarily heptapeptide components of the amino acid sequence:

(SEQ ID NO: 110)
Lys-X¹-Ser-U-X²-I-Lys

wherein X¹, U, X² and I are as hereinbefore defined.

Preferred conjugates of the invention comprising peptide components of SEQ ID No: 110 include those in which:
X¹ represents Hyp or, more preferably, Pro;
U represents DOPA or, more preferably, Tyr;
X² represents Pro or, more preferably, Hyp.
I represents Hyp or, more preferably, DOPA or Tyr.

In this respect, preferred conjugates of the invention comprising peptide components of SEQ ID No: 110 include those comprising the amino acid sequence:

(SEQ ID No: 111)
Lys-Pro-Ser-Tyr-Hyp-DOPA-Lys
and
(SEQ ID NO: 112)
Lys-Pro-Ser-Tyr-Hyp-Tyr-Lys

Particularly-preferred peptide sequences include those comprising the amino acid sequence:

(SEQ ID No: 1)
Ala-Lys-Pro-Ser-Tyr-Hyp-Hyp-Thr-DOPA-Lys;
SEQ ID No: 2)
Ala-Lys-Pro-Ser-Tyr-Hyp-Hyp-Thr-Tyr-Lys;
SEQ ID No: 12)
Ala-Lys-Pro-Ser-Tyr-Hyp-Thr-Tyr-Hyp-Lys;
SEQ ID No: 13)
Ala-Lys-Pro-Ser-Tyr-Hyp-Thr-DOPA-Hyp-Lys;
SEQ ID No: 104)
Ala-Lys-Pro-Ser-Tyr-Hyp-Thr-Tyr-Lys-Hyp;
SEQ ID No: 111)
Lys-Pro-Ser-Tyr-Hyp-DOPA-Lys;
and
SEQ ID NO: 112)
Lys-Pro-Ser-Tyr-Hyp-Tyr-Lys.

It should further be noted that heptapeptide compounds of the amino acid sequence SEQ ID No: 110 may be novel per se, and therefore useful as human and animal medicines in their own right, whether or not they are in the form of a conjugate of the invention.

Such compounds are also indicated as pharmaceuticals (and/or in veterinary science), including in any one of the indications mentioned hereinafter, in the form of any one of the pharmaceutical formulations mentioned hereinafter, and/or in any one of the combinations/kits of parts mentioned hereinafter. They may also be used as cosmetics and/or as part of a medical device.

According to a further aspect of the invention, there is provided a peptide of the amino acid sequence:

(SEQ ID NO: 110)
Lys-X¹-Ser-U-X²-I-Lys

wherein X¹, U, X² and I are as hereinbefore defined, or a regioisomer, a stereoisomer, or a pharmaceutically- or and/or a cosmetically-acceptable salt (as hereinbefore defined) thereof.

The skilled person will understand that a conjugate is a compound formed by electrostatically linking and/or covalently linking a chemical compound to a different chemical compound.

The term ‘electrostatic cross-linking’ will be understood by the skilled person to include the association of disordered molecules into an ordered state by virtue of its nature or by electrostatic interactions (also referred to as ‘self-assembly’, which is a primary mechanism of gelation observed in amphiphilic peptide molecules (Hauser et al., Biomed. Mat. 2015, 11, 014103).

In this case, conjugates of the invention are preferably formed by covalently linking one or more local anaesthetics to one or more of the peptide components as defined above.

In this respect, conjugates of the invention may comprise one or more local anaesthetic molecules.

Conjugates of the invention may feature at least one covalent bond (e.g. an amide bond) formed by a reaction between a carboxylic acid (i.e. —CO₂H) moiety present (e.g. at the C-terminus) of a peptide component as hereinbefore defined, and an amine (i.e. —NH₂) group that is present in the local anaesthetic molecule. For example, an amide bond may be formed between an carboxylic acid group of the C-terminal amino acid in the group Y, Y¹ I or J in a peptide component of SEQ ID Nos: 3 or 63 (as appropriate) and an amine group of a local anaesthetic.

As used herein, Pro represents proline, Ala represents alanine, Ser represents serine, Tyr represents tyrosine, Hyp represents hydroxyproline (including 3-hydroxyproline (3Hyp) and 4-hydroxyproline (4Hyp)), diHyp represents dihydroxyproline (including 3,4-dihydroxyproline (3,4diHyp), 3,5-dihydroxyproline (3,5diHyp) and 4,5-dihydroxyproline (4,5diHyp)), Thr represents threonine, Lys represents lysine, Ala represents alanine and DOPA represents 3,4-dihydroxyphenylalanine. 3,4-Dihydrocinnamic acid (HCA) residues are essentially DOPA residues but without the —NH₂ group in the 2- or α-carbon position relative to the carboxylic acid that is attached to the N-terminal amino acid (whether Lys or Ala).

Conjugates of the invention, whether in the form of salts or otherwise, include regioisomers within amino acids of the peptides (for example diHyp, Hyp and Tyr moieties), as well as mixtures of such regioisomers. For example, included within the definition of Tyr are, not only tyrosine (4-hydroxyphenylalanine), but also 2- and 3-hydroxyphenylalanine. Included within the definition of Hyp are 4-hydroxyproline (4Hyp), 3-hydroxyproline (3Hyp) and 5-hydroxyproline (5Hyp). It is more preferred that Hyp residues are 4-hydroxyproline. Similarly, included within the definition of diHyp are 3,4-dihydroxyproline (3,4diHyp), 3,5-dihydroxyproline (3,5diHyp) and 4,5-dihydroxyproline (4,5diHyp). It is more preferred that diHyp residues are 3,4-dihydroxyproline (3,4diHyp).

Also, in addition to the standard central carbon atom of the amino acids in the conjugates of the invention (which are normally but not exclusively in the L-configuration), certain amino acids in the sequence comprise further chiral carbon atoms. All such stereoisomers and mixtures (including racemic mixtures) thereof are included within the scope of the invention. In respect, included within the definition of Hyp are trans-4-hydroxy-L-proline, cis-4-hydroxy-L-proline, trans-3-hydroxy-L-proline, cis-3-hydroxy-L-proline, trans-5-hydroxy-L-proline and cis-5-hydroxy-L-proline, however we prefer that the Hyp that is employed in conjugates of the invention is 4-hydroxy-L-proline. Similarly, corresponding definitions may be applied to diHyp, in which the two hydroxy groups can also be cis or trans relative to each other. In any event, individual enantiomers of peptide components as hereinbefore defined that may form part of a conjugate of the invention are included within the scope of the invention.

Conjugates of the invention may be in the form of salts. Salts that may be mentioned include pharmaceutically-acceptable and/or cosmetically-acceptable salts, such as pharmaceutically- and/or cosmetically-acceptable acid addition salts and base addition salts. Such salts may be formed by conventional means, for example by reaction of a conjugate of the invention with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of the conjugate of the invention in the form of a salt with another counter-ion, for example using a suitable ion exchange resin.

Preferred salts include, for example, acetate, hydrochloride, bisulfate, maleate, mesylate, tosylate, alkaline earth metal salts, such as calcium and magnesium, or alkali metal salts, such as sodium and potassium salts. Most preferably, conjugates of the invention may be in the form of acetate salts.

Conjugates of the invention may be prepared by way of conventional techniques, for example by way of standard amino acid coupling techniques, using standard coupling reagents and solvents, for example as described hereinafter. Conjugates of the invention may be synthesised from available starting materials using appropriate reagents and reaction conditions. In this respect, the skilled person may refer to inter alia “Comprehensive Organic Synthesis” by B. M. Trost and I. Fleming, Pergamon Press, 1991.

Further references that may be employed include “Heterocyclic Chemistry” by J. A. Joule, K. Mills and G. F. Smith, 3 rd edition, published by Chapman & Hall, “Comprehensive Heterocyclic Chemistry II” by A. R. Katritzky, C. W. Rees and E. F. V. Scriven, Pergamon Press, 1996 and “Science of Synthesis”, Volumes 9-17 (Hetarenes and Related Ring Systems), Georg Thieme Verlag, 2006.

Conjugates of the invention may be isolated from their reaction mixtures and, if necessary, purified using conventional techniques as known to those skilled in the art. Thus, processes for preparation of conjugates of the invention as described herein may include, as a final step, isolation and optionally purification of the conjugate of the invention.

It will be appreciated by those skilled in the art that, in the processes described above and hereinafter, the functional groups of intermediate compounds may need to be protected by protecting groups. The protection and deprotection of functional groups may take place before or after a reaction.

Protecting groups may be applied and removed in accordance with techniques that are well-known to those skilled in the art and as described hereinafter. For example, protected compounds/intermediates described herein may be converted chemically to unprotected compounds using standard deprotection techniques. The type of chemistry involved will dictate the need, and type, of protecting groups as well as the sequence for accomplishing the synthesis. The use of protecting groups is fully described in ‘Protective Groups in Organic Synthesis’, 5th edition, T. W. Greene & P. G. M. Wutz, Wiley-Interscience (2014), the contents of which are incorporated herein by reference.

Conjugates of the invention are useful as human and animal medicine. They are therefore indicated as pharmaceuticals (and/or in veterinary science), although they may also be used as cosmetics and/or as part of a medical device.

Conjugates of the invention may also possess pharmacological activity as such, certain pharmaceutically-acceptable (e.g. ‘protected’) derivatives of conjugates of the invention may exist or may be prepared which may not possess such activity, but which may be administered and thereafter be metabolised or chemically transformed to form conjugates of the invention. Such compounds (which may possess some pharmacological activity, provided that such activity is appreciably lower than that of the active conjugates to which they are metabolised/transformed) may therefore be described as ‘prodrugs’ of conjugates of the invention.

As used herein, references to prodrugs will include compounds that form a conjugate of the invention, in an experimentally-detectable amount, within a predetermined time, following administration. All prodrugs of the conjugates of the invention are included within the scope of the invention.

When conjugates of the invention possess pharmacological activity, they are particularly useful in the treatment of inflammation and/or pain.

The term ‘treatment of inflammation’ includes the treatment of inflammation in any organ of the body (including soft tissue, joints, nerves, the vascular system, internal organs, mucosal surfaces and the skin), irrespective of the cause, and also includes all such inflammatory disorders or conditions, and/or disorders or conditions characterized by inflammation (e.g. as a symptom).

Inflammatory disorders and/or conditions may be (and are typically) characterized by activation of immune defence mechanisms, resulting in an effect that is more harmful than beneficial to the host. Such conditions are generally associated with varying degrees of tissue redness or hyperemia, swelling, edema, hyperthermia, pain (including aching), exudation of body fluids, itching (pruritis), cell death and tissue destruction, cell proliferation, and/or loss of function.

Inflammatory conditions that may be mentioned include arteritis, diabetes mellitus, metabolic syndrome, rosacea, asthma and allergy, ankylosing spondylitis, chronic obstructive pulmonary disease, gouty arthritis, inflammatory bowel disease (such as Crohn's disease and ulcerative colitis), multiple sclerosis, osteoarthritis, pancreatitis, prostatitis, psoriatic arthritis, rheumatoid arthritis, tendinitis, bursitis, Sjögren's syndrome, systemic lupus erythematosus, uveitis, urticaria, vasculitis, mastocytosis, diabetic vascular complications, migraine, atherosclerosis and associated cardiovascular disorders.

A disease state characterised by inflammation that may be mentioned is chronic obstructive pulmonary disease (COPD). A further disease state characterised by inflammation that may be mentioned is inflammatory bowel diseases including Crohn's disease and, especially, ulcerative colitis. Other disease states characterized by inflammation that may be mentioned are gynaecological diseases, such as cervicitis, vaginitis (e.g. radiation vaginitis) and colpitis. Diseases that affect the gastrointestinal tract, such as gastrohelcosis (e.g. gastritis, gastric ulcer, gastric cancer and other stomach mucosa diseases) as well as gastroesophageal reflux disease (GERD), constipation, and gastritis, inflammation associated with cancers and infections (e.g. viral infections, such as the common cold or influenza).

Inflammatory conditions that may be more especially mentioned include inflammations of the skin or mucosa (including the oral, nasal, ocular, vaginal, cervical and/or anorectal mucosae, more particularly the oral or nasal mucosae), such as inflammation resulting from infections (such as viral and/or bacterial infections), or allergic/atopic conditions (such as rhinitis (e.g. allergic rhinitis), pharyngitis, periodontitis, gingivitis, xerophthalmia, conjunctivitis (e.g. allergic conjunctivitis), dermatitis, urticaria (hives) and food allergy); and other inflammatory conditions, such as herpes, drug eruptions, polymorphous light eruptions, sunburn, early manifestations of skin cancers (erythema-like skin lesions), pathological hair loss (including following skin grafting), chemo rash, psoriasis, erythema multiforme, folliculitis, eczema and external otitis. A disease state that may be mentioned is polymorphous light eruptions.

More particularly, conjugates of the invention may be used to treat certain conditions characterized by inflammation, and/or with which inflammation is associated. Such conditions may include wounds (including abrasions (scratches), incisions (including operative incisions), lacerations, punctures, avulsions, bruising and scarring), and burns (including inflammation resulting from surgery following burns, such as skin grafting) and other conditions, such as hemorrhoids. Wounds may be acute or chronic, and/or may result from one or more inflammatory disorders as defined herein.

Wounds of the skin or mucosa may arise from internal or external physical injury to the membrane surface, or may be caused by (i.e. be a symptom of) an underlying physiological disorder.

Physical (e.g. ‘open’) wounds may be caused by sharp objects (cuts, incisions, punctures) or blunt objects/mechanical forces (lacerations, abrasions, avulsions), physical blows (bruises), heat or chemicals (burns and blisters), UV light (sunburn), cold (chilblains or frostbite). Wounds may be superficial (damage only to the epidermis and/or dermis) or may be full thickness wounds (damage below the epidermis and/or dermis). In serious cases, subcutaneous and/or submucosal tissues, such as muscles, bones, joints, and even internal organs, may be damaged.

Conjugates of the invention may also be useful in the suppression of the production of melanin pigmentation, which may or may not result from inflammation and/or wound healing. Conjugates of the invention may also be useful in the suppression of disorders associated with melanin pigmentation, such as chloasma, freckles, melanosis, malar rash and other chromatosis, skin cancers with melanoma, and chromatosis that is caused by exposure to the sun or skin diseases like acne.

Wounds may also arise as a consequence of (e.g. inflammatory) diseases or disorders. Such wounds may be terms ‘chronic wounds’ and may include blistering and/or ulcers of the skin and mucosa. These are common conditions that are often long-lasting and difficult to treat. Skin tissues can often be damaged, removed, liquefied, infected and/or necrotic. Ulcers can lead to secondary consequences to health particularly if they become infected, are hard to heal and are costly to treat. They can also cause significant psychological stress and economic loss to patients, affecting both general well-being and quality of life.

In the alternative, inflammatory skin conditions or diseases in which conjugates of the invention find particular utility include psoriasis, acne, eczema and dermatitis, especially allergic/atopic dermatitis, as well as in the treatment of mucosal inflammation as characterized by rhinitis, especially allergic rhinitis, hemorrhoids, chronic obstructive pulmonary disease and ulcerative colitis, for example.

Psoriasis is a chronic, inflammatory skin disease with a tendency to recur (some patients never heal during their entire life). Clinical manifestations of psoriasis mainly include erythema and scales. It can occur over the whole body, but is more commonly observed on the scalp and limbs.

Acne is a follicular (pilosebaceous unit) chronic, inflammatory skin disease, the occurrence of which is closely related to main factors like hypersteatosis, blocked pilosebaceous ducts (including closed and open comedones), bacterial infection and inflammatory reactions, that tends to occur during youth, characterized by multiform skin lesions on the face. The term acne thus includes regular acne and acne rosacea (i.e. copper nose).

Eczema is a skin inflammatory reaction with strong itching caused by a variety of internal and external factors. It has three phases, acute, sub-acute, and chronic. In the acute phase, there is a tendency for the production of exudates, while the chronic phase includes infiltration and hypertrophy. Skin lesions are often itchy and recur easily.

Dermatitis is a common skin disease characterized by coarseness, redness, itching, eczema, and dryness. Small lumps, refractory ulcers, and pigmented spots caused by dermatitis may, if not treated promptly, develop to basal cell carcinoma, squamous cell carcinoma, and malignant melanoma. Dermatitis may be caused by various internal and external infectious or non-infectious factors, including substances (contact dermatitis) or allergy (allergic/atopic dermatitis). Also included is seborrheic dermatitis (seborrheic eczema) and all forms of steroid-dependent dermatitis (including light-sensitive seborrheic, perioral dermatitis, rosacea-like dermatitis, steroid-rosacea, steroid-induced rosacea, rosacea, steroid dermatitis resembling rosacea, topical corticosteroid-induced rosacea-like dermatitis and, more particularly, facial corticosteroid addictive dermatitis (FCAD) or facial corticosteroid-dependent dermatitis (FCDD), as characterized by flushing, erythema, telangiectasia, atrophy, papules and/or pustules in the facial area after long-term treatment with (including uncontrolled use, abuse or misuse of) topical corticosteroids; see, for example, Xiao et al., J. Dermatol., 2015, 42, 697-702 and Lu et al., Clin. Exp. Dermatol., 2009, 35, 618-621).

Rhinitis is irritation and inflammation of the mucous membrane inside the nose. Common symptoms of rhinitis include a stuffy nose, runny nose, sneezing and post-nasal drip. The most common kind of rhinitis is allergic rhinitis, caused by an allergen, such as pollen, dust, mould, or flakes of skin from certain animals. It has been surprisingly found that patients with allergic rhinitis who were treated with conjugates of the invention experienced relief of eye itchiness, even when conjugates of the invention were administered nasally (i.e. to the nasal mucosa).

Hemorrhoids are swellings caused by inflammation of the hemorrhoidal blood vessels found inside or around the rectum and the anus. Symptoms include bleeding (i.e. wounding) after the passage of a stool, prolapse of the hemorrhoid, mucus discharge and itchiness, soreness, redness and swelling in the area of the anus. Hemorrhoids are believed to be a consequence of an increase of pressure in the abdomen, for example, as a result of constipation or diarrhea.

Chronic obstructive pulmonary disease (COPD) is the name for a group of lung conditions that cause breathing difficulties, including emphysema (damage to the alveoli) and chronic bronchitis (long-term inflammation of the airways). COPD occurs when the lungs become inflamed, damaged and narrowed. The damage to the lungs is usually irreversible and results in an impairment of the flow of air into and out of the lungs. Symptoms of COPD include breathlessness, productive cough, frequent chest infections and persistent wheezing. The most common cause of the disease is smoking, although other risk factors include high levels of air pollution and occupational exposure to dust, chemicals and fumes.

Conjugates of the invention may have positive effects in mitigating erythema, redness and swelling, edema, blisters, and bullous pemphigoid caused by various conditions including those mentioned generally and specifically herein, and may inhibit exudation of subcutaneous tissue fluid, and suppressing itching and pain caused by such inflammatory conditions.

Other inflammatory conditions that may be mentioned include:
(a) Mucosal inflammation, such as oral mucositis, aphthous ulcers, otitis media, laryngitis, tracheitis, esophagitis, gastritis, enteritis and enterocolitis (including bacillary dysentery, chronic amoebic dysentery, schistosomiasis, nonspecific ulcerative colitis and regional enteritis), cervicitis and endocervicitis, endometritis, inflammation caused by inhalation injury and the like, as well as mucosal inflammation associated with cancers, and infections (e.g. viral infections, such as the common cold or influenza), that affect mucosal surfaces, such as those in the oral cavity, the nasopharynx, the ear, the throat, the trachea, the gastrointestinal tract, the cervix, etc.
(b) Orthopedic inflammation associated with, for example bone fractures, pyogenic infection of bones and joints, inflammation caused by rheumatic bone diseases, as well as pyogenic osteomyelitis (acute, chronic, localized, sclerotic, post-traumatic), pyogenic arthritis; bone tumors (osteoma, osteoid osteoma, chondroma), bone cysts, osteoclastoma, primary bone sarcoma (osteosarcoma, chondrosarcoma, osteofibrosarcoma, Ewing's sarcoma, non-Hodgkin's lymphoma, myeloma, chordoma), metastatic bone tumors, tumor-like lesions of bone (bone cyst, aneurysmal bone cyst, eosinophilic granuloma, fibrous dysplasia); and rheumatic arthritis.
(c) Nerve inflammation, such as peripheral polyneuritis, facial neuritis, peripheral neuritis, subcutaneous neuritis, ulnar neuritis, intercostal neuritis, etc.
(d) Subcutaneous and submucosal soft tissue inflammation, such as myositis, ligamentitis, tendonitis, panniculitis capsulitis, lymphadenitis, bubonadentitis, tonsillitis, synovitis, fasciitis, and soft tissue inflammation caused by injuries, contusion or laceration of muscles, ligaments, fascia, tendons, membrana synovialis, fat, articular capsules, and lymphoid tissue.
(e) Vascular inflammation, such as allergic leukocytoclastic vasculitis, allergic cutaneous vasculitis, polyarteritis nodosa, thrombotic vasculitis, granulomatous vasculitis, lymphocytic vasculitis, vasculitis with abnormalities in blood composition, and rheumatic vasculitis, as well as vascular inflammation associated with vascular cancers caused by allergic leukocytoclastic vasculitis, polyarteritis nodosa, thrombotic vasculitis, granulomatous vasculitis, lymphocytic vasculitis, vasculitis with abnormalities in blood composition, and rheumatic vasculitis.
(f) Inflammation of the internal organs, such as the heart, stomach, intestine, lung, liver, spleen, kidney, pancreas, bladder, ovary, and prostate, including but not limited to pericarditis, myocarditis, endocarditis, pneumonia, hepatitis, splenitis, nephritis pancreatitis, cystitis, oophoritis, prostatitis and treatment of gastric ulcer.
(g) Inflammation of the eye and surrounding area, such as conjunctivitis, keratitis (e.g. acute epithelial keratitis, nummular keratitis, interstitial keratitis, disciform keratitis, neurotrophic keratitis, mucous plaque keratitis, herpes simplex keratitis, herpes zoster keratitis, bacterial keratitis, fungal keratitis acanthamoebic keratitis, onchocercal keratitis, superficial punctate keratitis, ulcerative keratitis, exposure keratitis photokeratitis and contact lens acute red eye), optic neuritis, etc.
(h) Inflammation of the gums and the oral cavity, such as periodontitis, gingivitis, dental ulcers, etc.
(i) Inflammation associated with rheumatism, such as rheumatic vasculitis, rheumatoid arthritis, rheumatic bone diseases, ankylosing spondylitis, bursitis, Crohn's disease, gout, infectious arthritis, juvenile idiopathic arthritis, osteoarthritis, osteoporosis, polymyalgia rheumatica, polymyositis, psoriatic arthritis, scleroderma, Sjögren's syndrome, spondyloarthropathies, systemic lupus erythematosus, tendinitis, etc.

Conjugates of the invention may in particular be used to relieve the pain (including aching) associated with inflammation and/or wounding.

In particular, conjugates of the invention may be used to relieve procedural pain and/or non-procedural pain. The skilled person will understand that the term ‘procedural pain’ (i.e. operation pain) refers to acute pain that is associated with medical investigations and treatments conducted for the purpose of healthcare. The term ‘non-procedural’ refers to general pain that is associated with inflammation and/or wounding (e.g. pain associated with dental ulcers, burns and/or scars), and is not a consequence of a particular medical intervention.

Conjugates of the invention may be used to treat not only the inflammation, pain (including aching) and/or pruritis (itching) associated with the wound itself and the healing process, but also to prevent the exudation of body fluids from wounds, the risk of infection, and the prevention of physiological reactions that result from inflammation and/or wound healing processes, such as scarring and melanin pigmentation.

Scarring is a consequence of inflammation and/or wound healing and is a general term for the formation of fibrotic tissue that is a consequence of such inflammation/healing.

Conjugates of the invention are therefore useful in the treatment or alleviation of pain, in any organ of the body, for example soft tissue, joints, nerves, the vascular system, internal organs, the skin and mucosal surfaces (e.g. the oral cavity, the pharynx, and pharyngeal mucosa).

Conjugates of the invention may also be used to provide pain relief and/or anaesthesia in the treatment of acute pain (before, during and/or after surgical procedures, diagnostic procedures and/or after traumas), and/or chronic pain (including post-surgical or post-traumatic pain), at and/or within any area of the body, by, for example, topical, transdermal, intradermal, transmucosal, subcutaneous and/or intramucosal administration, by infiltration, by brachial plexus block, by epidural (extradural) block, by spinal anesthesia (subarachnoid block) and/or by iontophoresis, any and all of which may be achieved by inter alia injection of a conjugate of the invention on a local basis and/or by other form of local and/or topical application of conjugate of the invention, prior to, during and/or after a surgical or a diagnostic procedure.

Surgical and diagnostic procedures will be understood to include general surgery or other surgical and/or diagnostic interventions, such as dental procedures (including restorative or cosmetic operations, fillings, crowns, root canal treatment or extractions); skin surgery, including laser surgery to treat melanin pigmentation and other cosmetic procedures, such as injection of e.g. hyaluronic acid, collagen and/or other cosmetic materials into the dermis or the epidermis; peripherial blood vessel surgery; podiatry (cutaneous, nail avulsions, matricectomy, bunionectomy and hammertoe repairs); surgery to mucosal surfaces, such as the nasal, rectal, colonic, oral and ocular mucosae, including eye surgery (e.g. cataract removal) and ear nose and throat operations (including head and neck surgery); shoulder and/or arm surgery; surgery to any joint in the human body; surgery to internal organs, including the heart, the lungs and/or the abdomen (including hernia repair); drainage of bodily fluids (e.g. ascites or hematomas); insertion of medical devices, including pacemakers, catheters, implantable defibrillators, drug implants and contraceptive devices (IUDs); venipuncture and intravenous cannula insertion; bone joint surgery (e.g. surgery of the pelvis, the hips and the legs); spinal procedures (surgery and lumbar punctures); gynaecological and urological procedures (including smear tests and cystoscopies); gastrointestinal endoscopies and colonoscopies; brochioscopies; intubation; and/or in interventions in obstetrics and/or childbirth.

Conjugates of the invention may be used in the treatment of painful diseases and/or conditions, such as stomatitis, oral mucositis (a common and often debilitating complication of cancer treatment); Burning Mouth Syndrome or Glossodynia; Sjögren's syndrome; xerostomia (the subjective complaint of dry mouth due to a lack of saliva); periodontitis (any inflammatory disease affecting the periodontium); toothache (odontalgia or odontalgy); throat infections and/or pharyngitis; canker sores and/or aphthous ulcers, to include any break in the mucous membrane, as well as painful diseases of the rectum (proctitis) and colon (e.g. colitis).

Assessment of pain alleviation may be determined by use of a VAS score. A VAS score is a scale of 0 to 10, wherein 0 is pain free and 10 is the worst imaginable pain. The need for pain relief is highly subjective, but may be generally manifest by an individual having a VAS score relating to pain that is at least 4 to 5, such as at least 6, for example at least 8.

In a further aspect of the invention, treatment may result in a decrease in the severity of symptoms corresponding to a decrease of score as measured according to VAS score herein of at least about 15%, such as least about 25%, more preferably at least about 30%, within about 10 minutes from administration of a composition comprising an effective dose of a conjugate of the invention. After about 30 minutes of such administration, the score may preferably be decreased by at least about 20%, such as at least about 30%, for example around about 40% to about 60%, more preferably at least about 40%, yet more preferably at least about 50%, and even more preferably at least about 60% after such administration. Withing about 1 hour of such administration, the VAS score may preferably result in a decrease of at least about 30%, preferably at least about 40%, more preferably at least about 50%, even more preferably at least about 55%, yet more preferably at least about 60%, even more preferably at least about 65%, and most preferably at least about 70%.

In addition, conjugates of the invention may also be used in the treatment of certain specific diseases of the digestive system, such as gastroesophageal reflux disease (GERD), which may be characterized by an acidic taste in the mouth, regurgitation, heartburn, pain with swallowing and/or sore throat, increased salivation (water brash), nausea, chest pain, and coughing. GERD may cause injury of the esophagus, including reflux esophagitis (i.e. inflammation of the esophageal epithelium which may cause ulceration at or around the junction of the stomach and esophagus), esophageal strictures (i.e. the persistent narrowing of the esophagus caused by reflux-induced inflammation), Barrett's esophagus (i.e. intestinal metaplasia (i.e. changes of epithelial cells from squamous to intestinal columnar epithelium of the distal esophagus) and/or esophageal adenocarcinoma (a form of cancer)).

Conjugates of the invention may also be used in the treatment of certain specific diseases of the respiratory system, such as pulmonary cystic fibrosis, usual interstitial pneumonia, allergic pneumonia, asbestosis, emphysema, pulmonary heart disease, pulmonary embolism, etc. A specific disease state that may be mentioned in idiopathic pulmonary fibrosis (IPF).

IPF is a diffuse and fatal pulmonary interstitial disease with pathological features including alveolar epithelial damage, massive proliferation of lung fibroblasts, excessive deposition of extracellular matrix, ultimately leading to irreversible lung tissue damage. In the latter stages of the disease, subjects with IPF experience respiratory failure and death. It has been found that conjugates of the invention may find utility in the treatment of IPF and/or alleviation of the symptoms associated with the disease.

Conjugates of the invention are particularly useful in the treatment of the following lung and/or fibrotic conditions (whether otherwise mentioned herein or not): lung fibrosis, renal fibrosis, liver fibrosis, silicosis, acute bronchitis, chronic bronchitis, tracheobronchitis, bronchial asthma, status asthmatics, bronchiectasis, upper respiratory tract infections (including the common cold and influenza), allergic airway inflammation, bacterial pneumonia, viral pneumonia, mycoplasma pneumonia, reckettsia, radiation pneumonia, pneumococcal (including staphylococcal, streptococcal and gram-negative bacillus) pneumonia, pulmonary candidiasis (including aspergillosis, mucormycosis, histoplasmosis, actinomycosis and nocardiosis), pulmonary mycosis, cryptococcosis, lung abscesses, anaphylactic pneumonia, extrinsic allergic alveolitis, pulmonary eosinophilia (including Loeffler's syndrome and eosinophilosis), obstructive pulmonary emphysema, pulmonary edema, pulmonary tuberculosis, respiratory alkalosis/acidosis, acute lung injury, interstitial lung disease, empyema, lung fibroma and cor pulmonale.

Particular mucosal disorders and disease in which conjugates of the invention find utility include anorectal diseases, such as diarrhea, hemorrhoids, abscesses, fistula, fissures, anal itching, anal sinusitis, warts and rectal prolapse; inflammatory bowel disease, including Crohn's disease and, particularly, ulcerative colitis; gynaecological diseases, such as cervicitis, vaginitis, pelvic pain and disorders; and dental diseases, such as paradentitis, for example.

Conjugates of the invention may further possess an antioxidation effect, by increasing SOD (superoxide dismutase) production and reducing lipid oxidation. Conjugates of the invention may therefore be considered to have antioxidant properties.

Conjugates of the invention may also possess antipyretic properties that allow for the treatment of a fever and/or alleviate the symptoms thereof; for example, by reducing a subject's body temperature, which results in a reduction of fever. Conjugates of the invention and formulations including them may therefore be considered to be antipyretics.

According to a further aspect of the invention there is provided a method of treatment of inflammation, of an inflammatory disorder, and/or of a disorder/condition characterised by inflammation (for example as a symptom), and/or a method of treatment of pain, which pain may or may not be associated with any of the foregoing, which method comprises the administration of a conjugate of the invention or a salt thereof to a patient in need of such treatment.

For the avoidance of doubt, in the context of the present invention, the terms ‘treatment’, ‘therapy’ and ‘therapy method’ include the therapeutic, or palliative, treatment of patients in need of, as well as the prophylactic treatment and/or diagnosis of patients which are susceptible to, inflammation and/or inflammatory disorders.

Conjugates of the invention may further possess antiviral properties that may allow for the treatment of a viral infection per se, that is treatment of a viral infection, or a viral disease, by interfering with the replication of the virus within a host, as opposed to the treatment of any symptoms of any viral infection or disease, such as pain and/or inflammation. Such antiviral properties may also allow for the prevention of the onset of such an infection or disease, the protection of cells in a host from (e.g. further) viral infection, prevention or arrest of the spread of viral infection or disease (within a single host, or from one host to a new host), or for the prevention of reactivation of a virus after latency in a host.

According to a further aspect of the invention there is provided a method of treatment of a viral infection, which method comprises the administration of a conjugate of the invention or a salt thereof to a patient in need of such treatment. Viral infections that may be mentioned include those caused by viruses in the following families: adenoviridae (e.g. adenovirus), papillomaviridae (e.g. human papillomavirus), polyomaviridae (e.g. BK virus; JC virus), herpesviridae (e.g. herpes simplex, type 1; herpes simplex, type 2; varicella-zoster virus; Epstein—Barr virus; human cytomegalovirus; human herpes virus, type 8), poxviridae (e.g. smallpox), hepadnaviridae (e.g. hepatitis B virus), parvoviridae (e.g. parvovirus B19), astroviridae (e.g. human astrovirus), caliciviridae (e.g. norovirus; Norwalk virus), picornaviridae (e.g. coxsackievirus, hepatitis A virus; poliovirus; rhinovirus), coronoviridae (e.g. severe acute respiratory syndrome (SARS) virus, including SARS-CoV-2), flaviviridae (e.g. hepatitis C virus; yellow fever virus; dengue virus; West Nile virus; tick-borne encephalitis virus), retroviridae (e.g. human immunodeficiency virus; HIV), togaviridae (e.g. rubella virus), arenaviridae (e.g. Lassa virus), bunyaviridae (e.g. hantavirus; Crimean-Congo hemorrhagic fever virus; Hantaan virus), filoviridae (e.g. Ebola virus; Marburg virus; Ravn virus), orthomyxoviridae (e.g. influenza viruses, including influenza A virus (e.g. H1N1 and H3N2 viruses), influenza B virus or influenza C virus), paramyxoviridae (e.g. measles virus; mumps virus; parainfluenza virus, respiratory syncytial virus), rhabdoviridae (e.g. rabies virus), hepeviridae (e.g. hepatitis E virus), reoviridae (e.g. rotavirus; orbivirus; coltivirus; Banna virus), as well as viruses not assigned to families, such as hepatitis D virus.

Viruses that may be more specifically mentioned include herpes simplex, type 1 and herpes simplex, type 2 viruses, human papillomavirus, influenza virus and parainfluenza virus.

Conjugates of the invention may further possess antibacterial and/or bacteriostatic properties that may allow for the treatment of a bacterial infection per se, that is treatment of a bacterial infection, or a bacterial disease, by interfering with bacterial growth or proliferation in a host, as opposed to the treatment of any symptoms of any bacterial infection or disease, such as pain and/or inflammation. Conjugates of the invention may therefore be considered to be bacteriocides and/or, preferably, bacteriostatic agents.

Such antibacterial properties may also allow for the prevention of the onset of such an infection or disease, the protection of cells in a host from (e.g. further) bacterial infection, prevention or arrest of the spread of bacterial infection or disease (within a single host, or from one host to a new host), or for the prevention of reactivation of a bacterium after latency in a host.

According to a further aspect of the invention there is provided a method of treatment of a bacterial infection, which method comprises the administration of a conjugate of the invention or a salt thereof to a patient in need of such treatment.

As disclosed herein, conjugates of the invention may further possess anticancer properties that may allow for the treatment of a cancer per se, that is treatment of a cancer by interfering with the cancer as opposed to the treatment of any symptoms of the cancer, such as pain and/or inflammation. Such anticancer properties may also include the prevention of the onset of such a disease e.g. by treating inflammation and thereby preventing such onset.

According to another aspect of the invention, there is provided a method of treatment of cancer, which method comprises the administration of a conjugate of the invention or a salt thereof to a patient in need of such treatment.

Particular cancers that may be mentioned include oral cancer, a nasopharynx cancer, a middle ear cancer, a conjunctival cancer, a throat cancer, a tracheal cancer, an esophageal cancer, a gastric cancer, an intestinal cancer, a cervical cancer, an endometrial cancer, skin cancer and the like caused by oral mucositis, rhinitis, otitis media, conjunctivitis, pharyngitis, laryngitis, tracheitis, esophagitis, gastritis, enterocolitis, cervicitis, endometritis, erythema-like skin lesions and the like. A particular skin cancer that may be mentioned is basal cell carcinoma.

‘Patients’ include reptilian, avian and, preferably, mammalian (particularly human) patients.

In accordance with the invention, conjugates of the invention comprising local anaesthetics are preferably administered locally, but conjugates of the invention comprising general anaesthetics may also be administered systemically, for example orally, intravenously or intraarterially (including by intravascular and other perivascular devices/dosage forms (e.g. stents)), intramuscularly, cutaneously, subcutaneously, transmucosally (e.g. sublingually or buccally), intramucosally, rectally, intravaginally, intradermally, transdermally, nasally, pulmonarily (e.g. tracheally or bronchially), preferably topically, or by any other parenteral route, in the form of a pharmaceutical preparation comprising the conjugate(s) in pharmaceutically acceptable dosage form(s).

Administration by inhalation (e.g. nasally) is particularly useful when the condition to be treated is rhinitis or inflammation resulting from viral infections of the airways (e.g. upper respiratory tract infections, such as the common cold and influenza).

Pulmonary administration is particularly useful when the condition to be treated is COPD or IPF. Topical forms of administration may be enhanced by creating a spray comprising the conjugates of the invention, e.g. by using a powder aerosol or by way of an aqueous mist using an appropriate atomisation technique or apparatus, such as a nebulizer.

Anorectal administration is particularly useful when the condition to be treated is hemorrhoids or ulcerative colitis, using an appropriate delivery means, such as a solution of foam to be injected or a suppository.

Administration to the lower gastrointestinal tract may also be achieved by parenteral, and particularly by peroral, delivery, by means of standard delayed- or extended-release coating techniques known to those skilled in the art. In particular, distinct parts of the upper or lower intestine may be targeted. For example, colonic administration can also be achieved by way of colon-targeted drug delivery means that are initially administered perorally or parenterally.

Conjugates of the invention, and especially those comprising general anaesthetics, may in the alternative be administered by direct systemic parenteral administration. Such administration may be useful in methods of treatment of an inflammatory and/or fibrotic disorder or condition of one or more internal organs of a patient.

Internal organs that may be mentioned include the stomach, the intestines, the pancreas, the liver, the spleen, the bladder, the vascular system, the ovaries, the prostate, preferably the heart and the kidneys and more preferably the lungs.

Fibrotic conditions of internal organs that may be mentioned include acute and/or severe internal fibrotic conditions characterised by the excessive accumulation of fibrous connective tissues (as described above) in and around inflamed or damaged tissues. Formulations of the invention may thus be useful in the treatment or prevention of fibrogenesis (as described above) and the morbidity and mortality that may be associated therewith. Thus, (e.g. acute and/or severe) fibrotic conditions of the internal organs that may be treated with formulations of the invention include fibrosis of the liver, the kidneys, the lungs, the cardiovascular system, including the heart and the vascular system, the pancreas, the spleen, the central nervous system (nerve fibrosis), bone marrow fibrosis, the eyes, the vagina, the cervix, etc.

Inflammatory conditions of internal organs include any condition that is, or may develop into a condition that is, severe (i.e. one that requires intensive medical treatment), and in which some sort of inflammatory component is apparent, as may be characterised by detectable inflammation, and further in which morbidity is manifested (or is expected) and/or is life-threatening.

Inflammatory conditions that may be mentioned include one or more acute disorders or conditions of internal organs (i.e. one or more conditions that require, or may develop into a condition that requires, immediate medical interventions) that are characterized by inflammation (e.g. as a symptom), such as acute internal injuries, in one or more internal organs (including any of the organs mentioned hereinbefore). By treating such acute inflammatory disorders, formulations of the invention may prevent or arrest the development of symptoms (acute or chronic) that are associated with such conditions, and also may arrest the progress of morbidity and/or mortality that is associated with such conditions.

Acute inflammatory conditions that may be mentioned thus include conditions such as peritonitis, pancreatitis, colitis, proctitis, gastritis, duodenitis, pharyngitis, GERD, parodontitis and stomatitis. Particular acute inflammatory conditions that may be mentioned include acute injury to one or more internal organs (including any of those mentioned hereinbefore), such as acute lung injury, inhalation injury (such as burns), acute respiratory distress syndrome (ARDS), severe acute respiratory syndrome (SARS), and multiple-organ inflammation, injury and/or failure.

Such conditions may be caused by internal or external trauma (e.g. injury or a burn), or by an infection by e.g. viruses, bacteria or fungi.

For example, proctitis (which includes eosinophilic, gonorrheal and/or ulcerative proctitis) may be caused by inflammatory bowel disease, infections, radiation (e.g. for cancer), drugs such as antibiotics, surgery or allergic conditions, such as food intolerances.

For example, multiple-organ inflammation, injury and/or failure may result from extensive and/or traumatic external injuries, including traumatic and/or extensive external burns. Traumatic external burns will be understood to include second-degree, and more particularly third-degree burns and fourth-degree, burns. Extensive external burns will be understood to include burns that affect at least about 10%, such as at least about 15%, including at least about 20% of a patient's body area. External (and internal) burns may result from exposure to heat, chemicals and the like.

Acute inflammatory and/or fibrotic conditions may also result from sepsis or septic shock, which can be caused by viral, bacterial or fungal infection. Furthermore, acute lung injury, ARDS and, particularly, SARS may be caused by viruses, such as coronaviruses, include the novel SARS coronavirus 2 (SARS-CoV-2).

Thus, in addition, one or more of the aforementioned (e.g. acute) inflammatory conditions may (indeed in some cases will likely) result in some form of internal tissue damage and/or dysfunction of relevant internal tissues. Relevant tissues thus include (e.g. mucosal) tissues, such as the respiratory epithelium. Such tissue damage may also give rise to one or more of the fibrotic conditions mentioned hereinbefore. For example, the SARS disease caused by the novel coronavirus SARS-CoV-2 (coronavirus disease 2019 or COVID-19) is known in many cases to result in fibrosis, which arise from one or more of a number of factors, including inflammation.

In this respect, conjugates of the invention and salts thereof find particular utility in the treatment of relevant inflammatory and/or fibrotic conditions on the basis that such conditions are often characterized by one or more comorbidities. By conditions that are ‘characterized by comorbidities’, we include that the main condition in question results in (or from) one more further medical conditions, including (and indeed preferably) those mentioned hereinbefore, at the same time, which conditions may interact and/or overlap with each other in some way.

In particular, however, when conjugates of the invention/salts thereof are administered directly and parenterally, they may be administered intravenously, intraarterially, intravascularly, perivascularly, intramuscularly, cutaneously, and/or subcutaneously, for example by way of direct injection, or by way of any other parenteral route, in the form of a conjugate of the invention or salt thereof in the form of a pharmaceutically-acceptable dosage form.

Pharmaceutically-acceptable formulations for use in injection (whether local (e.g. intradermally, intramucosally or subcutaneously) or systemic) may thus comprise conjugates of the invention in admixture with a pharmaceutically-acceptable adjuvant, diluent or carrier, which may be selected with due regard to the intended route of direct parenteral administration and standard pharmaceutical practice. Such pharmaceutically-acceptable carriers may be chemically inert to the active compounds and may have no detrimental side effects or toxicity under the conditions of use. Such pharmaceutically-acceptable carriers may also impart an immediate, or a modified, release of the conjugate of the invention.

Formulations for injection (whether for systemic, or local, e.g. intradermal, intramucosal, subcutaneous and/or intramuscular administration, or otherwise) may thus be in the form of an aqueous formulation such as an a suspension and/or, more preferably a solution (e.g. an (optionally) buffered aqueous formulation (e.g. solution), such as a physiological saline-containing formulation (e.g. solution), a phosphate-containing formulation (e.g. solution), an acetate-containing formulation (e.g. solution) or a borate-containing formulation (e.g. solution), or a freeze-dried powder that may be reconstituted with a vehicle, such as an aqueous vehicle prior to use (e.g. injection)).

Formulations for injection may include other suitable excipients known to those skilled in the art, such as solvents (e.g. water), co-solvents, solubilizing agents (e.g. cyclodextrins), wetting agents, suspending agents, emulsifying agents, thickening agents, chelating agents, antioxidants, reducing agents, antimicrobial preservatives, bulking agents and/or protectants.

Formulations for injection are preferably buffered by standard techniques to physiologically-acceptable pH values (e.g. pHs of between about 4.5 and about 9.5, e.g. about 6 and about 9, such as between about 6.5 and about 8.5) using buffers and/or pH modifiers as described herein, and/or may further comprise tonicity-modifying agents (such as sodium chloride).

The above notwithstanding, preferred modes of delivery of conjugates of the invention include topically to the site of inflammation (e.g. the mucosa, including the oral and/or nasal mucosa, the lung, the anorectal area and/or the colon or, more preferably, the skin) in an appropriate (for example pharmaceutically- and topically-acceptable) vehicle suitable for application to the skin and/or the appropriate mucosal surface, and/or a commercially-available formulation, but may also include oral, intravenous, cutaneous or subcutaneous, nasal, intramuscular, intraperitoneal, or pulmonary delivery.

Administration by injection is particularly useful for administering the conjugates of the invention, in the form of a solution of suspension into e.g. the dermis (e.g. intradermal or subcutaneous injection), the mucosa (e.g. intramucosal injection), a joint cavity or the eyes.

Administration by intradermal, subcutaneous and/or intramucosal injection is particularly useful for administering the conjugates of the invention, in the form of a solution or suspension (e.g. a dermal filler), into the dermis or the mucosa.

Administration by injection is particularly useful to fill, e.g. the surgical site of the nasal cavity, the anal fistula, the space between the gingival and the root or the sinus. This is particularly useful for shaping support and/or lubrication.

Conjugates of the invention will generally be administered in the form of one or more for example pharmaceutical formulations in admixture with a (e.g. pharmaceutically acceptable) adjuvant, diluent or carrier, which may be selected with due regard to the intended route of administration (e.g. topical to the relevant mucosa (including the lung) or, preferably, the skin) and standard pharmaceutical or other (e.g. cosmetic) practice.

Such pharmaceutically acceptable carriers may be chemically inert to the active compounds and may have no detrimental side effects or toxicity under the conditions of use. Such pharmaceutically acceptable carriers may also impart an immediate, or a modified, release of the conjugate of the invention.

Suitable pharmaceutical formulations may be commercially available or otherwise prepared according to techniques that are described in the literature, for example, Remington The Science and Practice of Pharmacy, 22^{nd} edition, Pharmaceutical Press (2012) and Martindale—The Complete Drug Reference, 38th Edition, Pharmaceutical Press (2014) and the documents referred to therein, the relevant disclosures in all of which documents are hereby incorporated by reference. Otherwise, the preparation of suitable formulations including conjugates of the invention may be achieved non-inventively by the skilled person using routine techniques.

Conjugates of the invention may be in the form of an aqueous formulation such as an emulsion, a suspension and/or a solution (e.g. an (optionally) buffered aqueous formulation (e.g. solution), such as a physiological saline-containing formulation (e.g. solution), a phosphate-containing formulation (e.g. solution), an acetate-containing formulation (e.g. solution) or a borate-containing formulation (e.g. solution)), or a freeze-dried powder.

Conjugates of the invention may further and/or in the alternative be combined with appropriate excipients to prepare:
gel formulations (for which suitable gel matrix materials include cellulose derivatives, carbomer and alginates, gummi tragacanthae, gelatin, pectin, carrageenan, gellan gum, starch, Xanthan gum, cationic guar gum, agar, noncellulosic polysaccharides, saccharides such as glucose, glycerin, propanediol, vinyl polymers, acrylic resins, polyvinyl alcohol, carboxyvinyl polymer and, particularly, hyaluronic acid);
lotions (for which suitable matrix materials include cellulose derivatives, glycerin, noncellulosic polysaccharides, polyethylene glycols of different molecular weights and propanediol);
pastes or ointments (for which suitable paste matrix materials include glycerin, vaseline, paraffin, polyethylene glycols of different molecular weights, etc.);
creams or foams (for which suitable excipients (e.g. foaming agents) include hydroxypropyl methyl cellulose, gelatin, polyethylene glycols of different molecular weights, sodium dodecyl sulfate, sodium fatty alcohol polyoxyethylene ether sulfonate, corn gluten powder and acrylamide);
powder aerosols (for which suitable excipients include mannitol, glycine, dextrin, dextrose, sucrose, lactose, sorbitol and polysorbates, e.g. a dry powder inhalant); liquid, for example, water (aerosol) sprays for oral use or for inhalation (for which suitable excipients include viscosity modifiers, such as hyaluronic acid, sugars, such as glucose and lactose, emulsifiers, buffering agents, alcohols, water, preservatives, sweeteners, flavours, etc.); and/or
injectable solutions or suspensions (which may be aqueous or otherwise and for which suitable excipients include solvents and co-solvents, solubilizing agents, wetting agents, suspending agents, emulsifying agents, thickening agents, chelating agents, antioxidants, reducing agents, antimicrobial preservatives, buffers and/or pH modifiers, bulking agents, protectants and tonicity-modifying agents), particular injectable solutions or suspensions that may be mentioned include dermal fillers (i.e. injectable fillers or soft-tissue fillers), particularly when the conjugate of the invention is combined with hyaluronic acid.

Moisturizing agents, such as glycerol, glycerin, polyethylene glycol, trehalose, glycerol, petrolatum, paraffin oil, silicone oil, hyaluronic acid and salts (e.g. sodium and potassium salts) thereof, octanoic/caprylic triglyceride, and the like; and/or antioxidants, such as vitamins and glutathione; and/or pH modifiers, such as acids, bases and pH buffers, may also be included in such formulations, as appropriate. Furthermore, surfactants/emulsifiers, such as hexadecanol (cetyl alcohol), fatty acids (e.g. stearic acid), sodium dodecyl sulfate (sodium lauryl sulfate), sorbitan esters (e.g. sorbitan stearate, sorbitan oleate, etc.), monoacyl glycerides (such as glyceryl monostearate), polyethoxylated alcohols, polyvinyl alcohols, polyol esters, polyoxyethylene alkyl ethers (e.g. polyoxyethylene sorbitan monooleate), polyoxyethylene castor oil derivatives, ethoxylated fatty acid esters, polyoxylglycerides, lauryl dimethyl amine oxide, bile salts (e.g. sodium deoxycholate, sodium cholate), lipids (e.g. fatty acids, glycerolipids, glycerophospholipids, sphingolipids, sterols, prenols, saccharolipids, polyketides), phospholipids, N,N-dimethyldodecylamine-N-oxide, hexadecyltrimethyl-ammonium bromide, poloxamers, lecithin, sterols (e.g. cholesterol), sugar esters, polysorbates, and the like; preservatives, such as phenoxyethanol, ethylhexyl glycerin, and the like; and thickeners, such as acryloyldimethyltaurate/VP copolymer, may be included. In particular, stearic acid, glyceryl monostearate, hexadecanol, sorbitan stearate, cetyl alcohol, octanoic/capric glyceride etc. may be included, particularly in cream formulations.

Conjugates of the invention, and (e.g. pharmaceutical) formulations (e.g. aqueous solutions, gels, creams, ointments, lotions, foams, pastes and/or dry powders as described above) including them, may further be combined with an appropriate matrix material to prepare a dressing or a therapeutic patch for application on a biological surface, such as the skin or a mucosal surface. Such formulations may thus be employed to impregnate a matrix material, such as gauze, non-woven cloth or silk paper. The therapeutic patch may alternatively be, for example, a band-aid, a facial mask, an eye mask, a hand mask, a foot mask, etc.

Vaseline may be employed for use in applying such dressings to wounds, but we have also found that ointments based on PEGs (e.g. PEG 400) may be combined with matrix materials to prepare dressings without the need to use Vaseline.

Conjugates of the invention may also be used in combination with solid supports (such as nasal dressings (for example, to stop nasal bleeding), dermal scaffolds (for example, in wound healing) or artificial bones (for example, in the case of bone grafting/implantation).

Conjugates of the invention may be administered for inhalation by way of suspension, a dry powder or a solution. Suitable inhalation devices include pressurized metered-dose inhalers (pMDIs), which may be hand- or breath-actuated and employed with or without a standard spacer device, dry powder inhalers (DPIs), which may be single-dose, multi-dose, and power-assisted, and soft mist inhalers (SMIs) or nebulizers, in which aerosol drug in a fine mist is delivered with slower velocity than a spray delivered using, for example, a pMDI.

In pMDIs, conjugates of the invention may be administered as a pressurized suspension of micronized particles distributed in a propellant (e.g. HFA, along with excipients, such as mannitol, lactose, sorbitol, etc.), or as an ethanolic solutions, to deliver one or more metered dose of between about 20 and about 100 μL with each actuation. Actuation may be effected by hand (e.g. pressing) or by inhalation (breath-actuation), involving a flow-triggered system driven by a spring.

In DPIs, conjugates of the invention may be administered in the form of micronized drug particles (of a size between about 1 and about 5 μm), either alone or blended with inactive excipient of larger particle size (e.g. mannitol), inside a capsule, which may be pre-loaded or manually loaded into the device. Inhalation from a DPI may de-aggregate the medication particles and disperse them within the airways.

In SMIs, conjugates of the invention may be stored as a solution inside a cartridge, which is loaded into the device. A spring may release the dose into a micropump, such that the dose is released when a button is pressed, releasing jet streams of drug solution.

Various nebulizers may also be used to administer conjugates of the invention in the form of a fine mist of aerosolized solution. Nebulizers may include breath-enhanced jet nebulizer (in which, with the assistance of a compressor, an air stream moves through jet causing drug solution to be aerosolized); breath-actuated jet nebulizers (in which, after a patient inhales, with the assistance of a compressor, an air stream moves through a tube causing the drug solution to be aerosolized); ultrasonic nebulizers (in which piezoelectric crystals vibrate causing aerosolization by heating causing nebulization); vibrating mesh nebulizers (in which piezoelectric crystals vibrate a mesh plate causing aerosolization to give very fine droplets without a significant change in temperature of the solution during nebulization).

According to a further aspect of the invention there is provided a process for the preparation of a pharmaceutical composition/formulation, as defined herein, which process comprises bringing into association a conjugate of the invention, as hereinbefore defined, with one or more pharmaceutically-acceptable excipient, as hereinbefore defined.

Conjugates of the invention may also be combined in treatment with one or more growth factors selected from platelet-type growth factors (including platelet-derived growth factors, PDGFs); osteosarcoma-derived growth factors (ODGF), epidermal growth factors (EGFs), transforming growth factors (TGFα and TGFβ), fibroblast growth factors (αFGF, βFGF), insulin-like growth factors (IGF-I, IGF-II), nerve growth factors (NGF), interleukin-type growth factors (IL-1, IL-1, IL-3), erythropoietin (EPO), and colony stimulating factor (CSF).

According to a further aspect of the invention there is provided a (e.g. pharmaceutical) composition comprising a conjugate of the invention and one or more pharmaceutically-acceptable excipient, such as an adjuvant, diluent or carrier. Preferred formulations that include conjugates of the invention comprising local anaesthetics are suitable for application locally to e.g. the mucosa (including the oral and/or nasal mucosa, the lung, the anorectal area and/or the colon) or, more preferably, the skin and therefore comprise a topically-, intradermally-, subcutaneously- and/or intramucosally-acceptable adjuvant, diluent or carrier.

There is, thus, further provided pharmaceutical compositions comprising conjugates of the invention that are suitable for, adapted for, and/or packaged and presented for topical administration (e.g. to the mucosa, including the oral and/or nasal mucosa, the lung, the anorectal area and/or the colon, or to the skin), as well as the use of such a formulation in the treatment of a disorder including inflammation, an inflammatory disorder and/or a condition characterized by inflammation (e.g. as a symptom), and the use of such a formulation in the treatment of pain (whether associated with inflammation or otherwise), by way of direct topical administration of that formulation (e.g. to the mucosa, including the oral and/or nasal mucosa, the lung, the anorectal area and/or the colon, or, preferably, to the skin), and/or by intradermal, subcutaneous and/or intramucosal injection.

For the avoidance of doubt, topical formulations comprising conjugates of the invention may be used in any and all conditions described herein, including treatments of inflammation, in the treatment of any and all inflammatory disorder(s), and/or in the treatment of any and all condition(s) characterized by inflammation, and/or of pain, as hereinbefore mentioned, defined or described. Similarly, topical formulations comprising conjugates of the invention that may be mentioned include any and all of those mentioned, defined or described herein. Any and all of the relevant disclosures herein are hereby incorporated by reference in conjunction with this aspect of the invention.

Topical (e.g. liquid- or (e.g. aqueous) solution-based) compositions comprising conjugates of the invention may be particularly useful in wound recovery, and may alleviate pain (including aching) and, particularly, pruritis/itching that is associated with the wound itself and the wound healing process. Such topical formulations comprising conjugates of the invention may be particularly useful in the prevention and/or suppression of the exudation of body fluids from wounds, particularly during the acute inflammation stage, for example during the first 48 hours, after a burn or wound has been inflicted. This prevents the risk of infection, and other physiological reactions. Such topical formulations comprising conjugates of the invention may also be particularly useful in the prevention and/or suppression of scarring and melanin pigmentation (vide supra), whether associated with wounds or otherwise.

Injectable (e.g. liquid- or (e.g. aqueous) solution-based) compositions comprising conjugates of the invention may be particularly useful in the treatment of local inflammation and/or the alleviation of pain, for example pain associated with any organs of the body, including soft tissue, joints, nerves, the vascular system, internal organs, the skin and mucosal surfaces, and providing local anaesthesia to treat acute pain (during or after surgical or diagnostic procedures or a trauma) and/or chronic pain (including post-traumatic pain) within any area of the body as hereinbefore described.

Administration of the conjugates of the invention may be continuous or intermittent. The mode of administration may also be determined by the timing and frequency of administration, but is also dependent, in the case of the therapeutic treatment of inflammation, on the severity of the condition.

Depending on the disorder, and the patient, to be treated, as well as the route of administration, conjugates of the invention may be administered at varying therapeutically effective doses to a patient in need thereof.

Similarly, the amount of the conjugate of the invention in a formulation will depend on the severity of the condition, and on the patient, to be treated, but may be determined by the skilled person.

In any event, the medical practitioner, or other skilled person, will be able to determine routinely the actual dosage, which will be most suitable for an individual patient, depending on the severity of the condition and route of administration. The dosages mentioned herein are exemplary of the average case; there can, of course, be individual instances where higher or lower dosage ranges are merited, and such are within the scope of this invention.

Doses may be administered between once and four (e.g. three) times daily.

Appropriate concentrations of conjugates of the invention in an aqueous solution product may be about 0.01 (e.g. about 0.1) to about 15.0 mg/mL, in all cases calculated as the free (non-salt) conjugate.

Appropriate topical doses of conjugates of the invention are in the range of about 0.05 to about 50 μg/cm² of treated area, such as about 0.1 (e.g. about 0.5) to about 20 μg/cm² of treated area, including about 1 to about 10 μg/cm² of treated area, such as about 5 μg/cm² of treated area, in all cases calculated as the free (non-salt) conjugate.

Appropriate doses of conjugates of the invention for inhalation are in the range of about 0.01 μg to about 2000 mg, for example between about 0.1 μg to about 500 mg, or between 1 μg to about 100 mg. Particular doses for nasal administration that may be mentioned include between about 10 μg to about 1 mg, particularly a dose of about 0.1 mg (i.e. about 100 μg). Nasal administration of about 0.1 mg per day of conjugates of the invention has been found to be particularly effective in the treatment of conditions associated with inflammation of the nasal passages and mucosae, such as rhinitis (e.g. allergic rhinitis) and/or conditions associated with nasosinusitis surgery.

Appropriate doses of conjugates of the invention for pulmonary administration (e.g. by inhalation) are in the range of about 0.01 μg to about 2000 mg, for example between about 0.1 μg to about 500 mg, or between 1 μg to about 100 mg. Particular doses for pulmonary administration that may be mentioned include between about 10 μg to about 10 mg, particularly a dose of about 0.6 mg (i.e. 60 μg) to 6 mg (e.g. for use in treating COPD or IPF).

We prefer that pH values of formulations comprising conjugates of the invention are in the range of about 1.0 to about 9.0 (for example about 3.0 to about 8.0).

In any event, the dose administered to a mammal, particularly a human, in the context of the present invention should be sufficient to effect a therapeutic response in the mammal over a reasonable timeframe (as described hereinbefore). One skilled in the art will recognize that the selection of the exact dose and composition and the most appropriate delivery regimen will also be influenced by inter alia the pharmacological properties of the formulation, the nature and severity of the condition being treated, and the physical condition and mental acuity of the recipient, as well as the age, condition, body weight, sex and response of the patient to be treated, and the stage/severity of the disease, as well as genetic differences between patients.

Conjugates of the invention are useful in human and animal medicine. In this respect, and as described above, conjugates of the invention that possess an appropriate degree of relevant pharmacological (or biological) activity per se may be used as human, and/or animal, medicines.

Conjugates of the invention may be combined with a multitude of known pharmaceutically-active ingredients, including any agent, or drug, capable of producing some sort of physiological effect (whether in a therapeutic or prophylactic capacity against a particular disease state or condition) in a living subject, including, in particular, mammalian and especially human subjects (patients). This is the case irrespective of whether the conjugate of the invention is employed:
as a separate pharmaceutically-active ingredient per se in combination therapy;
as, or as part of, a medical device;
as, or as the medical device part of, a drug-medical device combination; or even as a pharmaceutically-acceptable excipient.

Such patients may also (and/or may already) be receiving therapy based upon administration of one or more of such other, known pharmaceutically-active ingredients, to treat one or more of the conditions described herein, by which we mean receiving a prescribed dose of one or more of the active ingredients mentioned herein, prior to, in addition to, and/or following, treatment with a conjugate of the invention.

Pharmaceutically-active agents that may be co-administered with a conjugate of the invention include any agent, or drug, that is capable of producing some sort of physiological effect (whether in a therapeutic or prophylactic capacity against a particular disease state or condition) in a living subject, including, in particular, mammalian and especially human subjects (patients).

Pharmaceutically-active agents may, for example, be selected from anti-inflammatory agents, pro-inflammatory agents, antibiotics, anti-bacterial and/or antiprotozoal agents, antiviral agents (e.g. protease inhibitors), anaesthetics, sedatives, muscle relaxants and and wound recovery drugs (e.g. growth factors).

Non-limiting examples of anti-inflammatory drugs which may be used also include those used in the treatment of rheumatic diseases and/or arthritis (such as cataflam, betamethasone, naproxen, cyclosporin, chondroitin, celecoxib, etodolac, meclofenamate, salsalate, methylprednisolone, and piroxicam); osteoarthritis (such as sulindac, meloxicam, fenoprofen, etoricoxib, and nabumetone); inflammation and its symptoms, e.g. fever, pain, itchiness and/or swelling (such as mefenamic acid, indomethacin, aspirin, ketorolac, fluorometholone, loteprednol, hydrocortisone, fluorometholone, bromfenac, prednisolone acetate, indomethacin, and ibuprofen); allergies and their symptoms (such as pheniramine, diphenhydramine, naphazoline, antazoline, prednisolone, lodoxamide, pemirolast, oxymetazoline, ketotifen, naphazoline, emestine fumarate, olopatadine, azelastine, tranilast, levocabastine, cortisone, ephedrine, cetirizine, levocetirizine, pseudophedrine, fexofenadine, terfenadine, loratadine, and alexis); respiratory diseases, including asthma and/or COPD (such as budesonide, ciclesonide, nedocromil, dexamethasone, ambroxol, and pranlukast); skin diseases (such as mometasone, triamcinolone, desonide, sulfacetamide, tacrolimus, allantoin, and triamcinolone); mastocytosis (such as cromolyn); gout (such as diclofenac, and febuxostat); conjunctivitis (such as hydrobenzole, pranoprofen, and zinc sulfate); eye diseases (such as dextran 70, thyroxine/liothyronine, and ocular extractives), known or commercially-available pharmaceutically acceptable salts of any of the foregoing, and combinations of any of the forgoing compounds and/or salts.

Antiinflammatory drugs that may be used along with conjugates of the invention include anticholinergic agents, such as atropine sulfate, scopolamine and glycopyrrolate, as well as endogenous (and/or exogenous) lipid-based pro-resolving, antiinflammatory molecules or mediators, such as lipoxins, resolvins, and protectins. Pro-inflammatory agents that may be mentioned include prostaglandins (e.g. latanoprost, prostaglandin E1, and prostaglandin E2), and leukotrienes (e.g. Leukotriene B4).

Conjugates of the invention may in the alternative be administered along with one or more of the anaesthetics or sedatives described herebefore, such as opioids. However, the following opioids have a longer onset and duration of action and are frequently used for post-operative pain relief: buprenorphine, butorphanol, diamorphine, hydromorphone, levorphanol, pethidine, methadone, morphine, nalbuphine, oxycodone, oxymorphone and pentazocine

Additionally, muscle relaxants, which do not render patients unconscious or relieve pain may be used in conjunction with a conjugate of the invention for example after a patient has been rendered unconscious to facilitate intubation or surgery by at least partial paralysis. Appropriate agents in this regard include depolarizing muscle relaxants, such as succinylcholineand decamethonium, short-acting non-depolarizing muscle relaxants, such as mivacurium, rapacuronium, intermediate-acting non-depolarizing muscle relaxants, such as atracurium, cisatracurium, rocuronium and vecuronium, and long-acting non-depolarizing muscle relaxants, such as alcuronium, doxacurium, gallamine, metocurine, pancuronium, pipecuronium and tubocurarine.

Non-limiting examples of anti-bacterial drugs which may be used also include chloramphenicol, ofloxacin, levofloxacin, tobramycin, norfloxacin, ciprofloxacin, lomefloxacin, lincomycin, fluconazole, enoxacin, furazolidone, nitrofurazone, rifampicin, micronomicin, gentamicin, cetylpyridinium, neomycin, roxithromycin, sulfadiazine silver, clarithromycin, clindamycin, metronidazole, azithromycin, mafenide, sulfamethoxazole, paracetamol, chloramphenicol, pseudoephedrine, mupirocin, amoxicillin, amoxicillin/clavulanic acid, trimethoprim/sulfamethoxazole, cefalexin, moxifloxacin, known or commercially-available pharmaceutically acceptable salts of any of the foregoing, and combinations of any of the foregoing compounds and/or salts.

Non-limiting examples of antiviral drugs which may be used also include tobramycin ribavirin, acyclovir, moroxydine, foscarnet, ganciclovir, idoxuridine, trifluridine, brivudine, vidarabine, entecavir, telbivudine, foscarnet, zidovudine, didanosine, zalcitabine, stavudine, lamivudine, abacavir, emtricitabine, nevirapine, delavirdine, efavirenz, etravirine, rilpivirine, saquinavir, ritonavir, indinavir, nelfinavir, amprenavir, lopinavir, ritonavir, atazanavir, fosamprenavir, tipranavir, darunavir, telaprevir, boceprevir, simeprevir, asunaprevir, raltegravir, elvitegravir, dolutegravir, rsv-igiv, palivizumab, docosanol, enfuvirtide, maraviroc, vzig, varizig, acyclovir, ganciclovir, famciclovir, valacyclovir, penciclovir, valganciclovir, cidofovir, tenofovir disoproxil fumarate, adefovir dipivoxil, fomivirsen, podofilox, imiquimod, sinecatechins, interferon-α 2b (recombinant, human), known or commercially-available pharmaceutically acceptable salts of any of the foregoing, and combinations of any of the foregoing compounds and/or salts.

Non-limiting examples of wound recovery drugs which may be used also include basic fibroblast growth factor (recombinant, human; recombinant, bovine), epidermal growth factor (recombinant, human; yeast), rhEFG (I), acidic fibroblast growth factor (recombinant, human), granulocyte macrophage stimulating factor (recombinant, human), sulfadiazine silver, sulfadiazine zinc, fusidic Acid, bacitracin, chlorhexidine, silver nitrate, triethanolamine, ethacridine, retinoids, calf blood deproteinized extract, carraghenates, amiotide and known or commercially-available pharmaceutically acceptable salts of any of the foregoing, and combinations of any of the foregoing compounds and/or salts.

Such pharmaceutically-active ingredients include those that may be administered topically, e.g. to the skin or to a mucosal surface along with a conjugate of the invention. In this respect, preferred active ingredients from the above list include cyclosporin, chondroitin, loteprednol, fluorometholone, bromfenac, prednisolone acetate, indomethacin, oxymetazoline, ketotifen, naphazoline, emestine fumarate, olopatadine, azelastine, tranilast, levocabastine, cortisone, ephedrine, cetirizine, pseudoephedrine, levocetirizine, fexofenadine, terfenadine, loratadine, alexis, dexamethasone, ambroxol), sulfacetamide, tacrolimus, allantoin, triamcinolone, cromolyn, nedocromil, diclofenac, hydrobenzole, pranoprofen, zinc sulfate, dextran 70, thyroxine/liothyronine, ocular extractives, chloramphenicol, ofloxacin, levofloxacin, tobramycin, norfloxacin, ciprofloxacin, lomefloxacin, lincomycin, fluconazole, enoxacin, furazolidone, nitrofurazone, rifampicin, micronomicin, gentamicin, cetylpyridinium, neomycin, roxithromycin, sulfadiazine silver, clarithromycin, sulfamethoxazole, chloramphenicol, tobramycin ribavirin, acyclovir, moroxydine, foscarnet, ganciclovir, interferon-α 2b (recombinant, human), articaine, dextropropoxyphene, sevoflurane, cophenylcaine, lidocaine, prilocaine, pramoxine, benzocaine, dibucaine, diclonine, tetracaine, bupivacaine, basic fibroblast growth factor (recombinant, human; recombinant, bovine), epidermal growth factor (recombinant, human; yeast), rhEFG (I), acidic fibroblast growth factor (recombinant, human), granulocyte macrophage stimulating factor (recombinant, human), sulfadiazine silver, sulfadiazine zinc, fusidic acid, bacitracin, chlorhexidine, silver nitrate, triethanolamine, ethacridine, retinoids, calf blood deproteinized extract, carraghenates, amiotide, and known or commercially-available pharmaceutically acceptable salts of any of the foregoing, and combinations of any of the foregoing compounds and/or salts.

Other pharmaceutically-active ingredients that may be co-administered with a conjugate of the invention include those that may be administered to treat one or of the gastrointestinal disorders mentioned hereinbefore.

Non-limiting examples of gastrointestinal drugs include oxalazine (olsalazine), sulfasalazine, domperidone, erythromycin, berberine, dexamethasone, cefuroxime axetil, levofloxacin, mesalazine, belladonna, sulfobenzidine, azathioprine, sulfasalazine, live bacillus (such as Clostridium butyricum, licheniformis, cereus), probiotics (such as bifidobacterium) tegafur, nifuratel, amoxicillin, ampicillin, nystatin, allicin, cefadroxil, dyclonine, carmofur, fluorouracil, mosapride, sodium carbosulfan, thrombin, pantoprazole, cimetidine, cisapride, ethylenediamine diacetamine, nimustine, famotidine, barium sulfate, aminocaproic acid, roxatidine acetate, vincristine, azasetron, lentinan, bismuth salts (e.g. aluminate, potassium citrate) in combination with e.g. magnesium salts, magnesium trisilicate, bicarbonate, vitamin U, aluminium hydroxide, belladonna extract, famotidine and calcium carbonate, magnesium hydroxide, hydrotalcite, proton pump inhibitors (such as omeprazole, lansoprazole, rabeprazole, pantoprazole, dexlansoprazole or esomeprazole), glycine, trypsin, allantoin aluminium hydroxide, sodium L-glutamine gualenate, rebampette, rotundine, quxipite, lafutidine, thymus protein, hericium erinaceus, irsogladine maleate, nizatidine, L-glutamine and sodium azulene sulfonate (sodium gualenate), ranitidine, bismuth citrate, lactobacillin, bisacordine, dimethylsiloxane, live Clostridium butyricum, loperamide hydrochloride, dibazol, secnidazole, zinc acephate, montmorillonite, tegafur/gimeracil/oteracil, famotidine, oteracil, doxifluridine, capecitabine and known or commercially-available pharmaceutically acceptable salts of any of the foregoing.

Pharmaceutically-active ingredients that may be mentioned for use in combination with conjugates of the invention include active ingredients that are useful in the treatment of inflammation and/or inflammatory disorders (other anti-inflammatory agents).

Anti-inflammatory agents that may be used in combination with conjugates of the invention in the treatment of inflammation include therapeutic agents that are useful in the treatment of inflammation and/or of diseases characterized by inflammation as one of its symptoms, including those described hereinbefore. Depending on the condition to be treated, such anti-inflammatory agents may include NSAIDs (e.g. aspirin), aminosalysates (e.g. 5-aminosalicyclic acid (mesalazine)), leukotriene receptor antagonists (e.g. montelukast, pranlukast, and zafirlukast), corticosteroids, analgesics and certain enzymes, such as trypsin, for example as described hereinafter. Conjugates of the invention may also be combined with leukotrienes (e.g. cysteinyl leukotrienes, and leukotriene B4).

Other preferred agents that may be combined with conjugates of the invention include LTB4 (to treat wounds and burns), NSAIDS (e.g. aspirin) or montelukast (to treat inflammation generally) and trypsin (to treat inflammation of the mucosa associated with e.g. viral infections).

Conjugates of the invention may also be combined with other therapeutic agents which, when administered, are known to give rise to inflammation as a side-effect.

Conjugates of the invention may also be combined with stem cells (e.g. totipotent (omnipotent), pluripotent (such as embryonic or induced pluripotent stem cells), multipotent (such as mesenchymal stem cells), oligopotent (such as hematopoietic stem cells), or unipotent (such as muscle stem cells)).

Other known pharmaceutically-active ingredients may also be administered in combination with conjugates of the invention in numerous ways.

For example, conjugates of the invention may be ‘combined’ with the (or with the other) pharmaceutically-active ingredients (or ‘therapeutic agents’) for administration together in the same (e.g. pharmaceutical) formulation, or administration separately (simultaneously or sequentially) in different (e.g. pharmaceutical) formulations.

Thus, such combination products provide for the administration of conjugates of the invention in conjunction with the (or with the other) therapeutic agent, and may thus be presented either as separate formulations, wherein at least one of those formulations comprises a conjugate of the invention, and at least one comprises the (or the other) therapeutic agent, or may be presented (i.e. formulated) as a combined preparation (i.e. presented as a single formulation including a conjugate of the invention and the (or the other) therapeutic agent).

Thus, there is further provided:
(1) a (e.g. pharmaceutical) formulation including a conjugate of the invention; another pharmaceutically-active ingredient; and, optionally, a pharmaceutically-acceptable inactive excipient (e.g. adjuvant, diluent or carrier), which formulation is hereinafter referred to as a ‘combined preparation’; and
(2) a kit of parts comprising components:
(A) a conjugate of the invention, optionally in the form of an (e.g. pharmaceutical) formulation in admixture with a pharmaceutically-acceptable inactive excipient (e.g. adjuvant, diluent or carrier); and
(B) another pharmaceutically-active ingredient, optionally in the form of a (e.g. pharmaceutical) formulation in admixture with a pharmaceutically-acceptable adjuvant, diluent or carrier, which components (A) and (B) are each provided in a form that is suitable for administration in conjunction with the other.

In a further aspect of the invention, there is provided a process for the preparation of a combined preparation (1) as hereinbefore defined, which process comprises bringing into association a conjugate of the invention, the other pharmaceutically-active ingredient, and at least one (e.g. pharmaceutically-acceptable) excipient.

In a further aspect of the invention, there is provided a process for the preparation of a kit-of-parts (2) as hereinbefore defined, which process comprises bringing into association components (A) and (B). As used herein, references to bringing into association will mean that the two components are rendered suitable for administration in conjunction with each other.

Thus, in relation to the process for the preparation of a kit-of-parts as hereinbefore defined, by bringing the two components ‘into association with’ each other, we include that the two components of the kit-of-parts may be:
(i) provided as separate formulations (i.e. independently of one another), which are subsequently brought together for use in conjunction with each other in combination therapy; or
(ii) packaged and presented together as separate components of a ‘combination pack’ for use in conjunction with each other in combination therapy.

Thus, there is further provided a kit of parts comprising:
(I) one of components (A) and (B) as defined herein; together with
(II) instructions to use that component in conjunction with the other of the two components.

In relation to kits of parts described above, although the conjugate of the invention may be provided in the form of a (e.g. pharmaceutical) formulation, in admixture with one or more additional pharmaceutically-acceptable excipients (e.g. adjuvants, diluents or carriers), if the compound of the invention is provided with a view to it primarily performing its function as a medical device or as an excipient, it may not be provided along with such additional pharmaceutically-acceptable excipients. In any event, it is preferred that the (other) pharmaceutically-active ingredient of the kit of parts is provided in the form of a pharmaceutical formulation in admixture with a pharmaceutically-acceptable adjuvant, diluent or carrier.

The kits of parts described herein may comprise more than one (e.g. formulation including an) appropriate quantity/dose of a conjugate of the invention, and/or more than one (e.g. formulation including an) appropriate quantity/dose of the other pharmaceutically-active ingredient, in order to provide for repeat dosing. If more than one formulation comprising or quantity/dose of either of the foregoing is present, such may be the same, or may be different in terms of the dose of either compound, chemical composition(s) and/or physical form(s).

With respect to the kits of parts as described herein, by ‘administration in conjunction with’, we include that respective components are administered, sequentially, separately and/or simultaneously, over the course of treatment of the relevant condition.

Thus, in respect of the combination product according to the invention, the term ‘administration in conjunction with’ includes that the two components of the combination product (conjugate of the invention and other pharmaceutically-active ingredient) are administered (optionally repeatedly), either together, or sufficiently closely in time, to enable a beneficial effect for the patient, that is greater, over the course of the treatment of the relevant condition, than if either the conjugate of the invention, or (e.g. a formulation comprising) the other agent, are administered (optionally repeatedly) alone, in the absence of the other component, over the same course of treatment. Determination of whether a combination provides a greater beneficial effect in respect of, and over the course of treatment of, a particular condition will depend upon the condition to be treated or prevented, but may be achieved routinely by the skilled person.

Further, in the context of a kit of parts according to the invention, the term ‘in conjunction with’ includes that one or other of the two components may be administered (optionally repeatedly) prior to, after, and/or at the same time as, administration of the other component. When used in this context, the terms ‘administered simultaneously’ and ‘administered at the same time as’ include that individual quantities/doses of the relevant conjugate of the invention and other active pharmaceutical ingredient are administered within 48 hours (e.g. 24 hours) of each other.

Depending on the disorder, and the patient, to be treated, as well as the route of administration, conjugates of the invention may be administered at varying therapeutically effective doses to a patient in need thereof.

In relation to combined preparations and kits of parts described above, it is preferred that the other pharmaceutically-active ingredient is an anti-inflammatory (and/or pain-relieving) agent, or agent known to give rise to inflammation as a side-effect, as hereinbefore described.

Wherever the word ‘about’ is employed herein, for example in the context of amounts, such as concentrations and/or doses of the conjugates of the invention and/or the pharmaceutically-active ingredients, molecular weights or pHs, it will be appreciated that such variables are approximate and as such may vary by ±10%, for example ±5% and preferably ±2% (e.g. ±1%) from the numbers specified herein. In this respect, the term ‘about 10%’ means e.g. ±10% about the number 10, i.e. between 9% and 11%.

The conjugates, uses and methods described herein may also have the advantage that, in the treatment of the conditions mentioned hereinbefore, they may be more convenient for the physician and/or patient than, be more efficacious than, be less toxic than, have a broader range of activity than, be more potent than, produce fewer side effects than, or that it/they may have other useful pharmacological properties over, similar compounds or methods (treatments) known in the prior art, whether for use in the treatment of inflammation, inflammatory disorders, or disorders characterised by inflammation as a symptom (including wounds), or otherwise.

The invention is illustrated, but in no way limited, by the following examples

### EXAMPLES

### Example 1

(SEQ ID NO: 102)
Ala-Lys-Pro-Ser-Tyr-Hyp-Thr-Tyr-Hyp-Lys-Procaine

Fmoc-Lys(Boc)-CTC resin (7.35 g, R201005, USUN Pharma, Jiangsu, China) was loaded into a glass reaction column.

Methylene chloride (DCM, 200 mL; Shandong Jinling Chemical Industry Co. Ltd., Shandong, China) was added to the column and allowed to soak the resin for about half an hour. The DCM was then removed by vacuum filtration.

The resin was washed 3 times with N,N-dimethylformamide (DMF, 200 mL; Shandong shitaifeng Fertilizer Industry Co Ltd, Shandong, China).

A 20% piperidine solution in DMF (200 mL; Shandong shitaifeng Fertilizer Industry Co. Ltd, Shandong, China) was added as deprotection solution and reacted for 20 minutes. The solution was then removed by vacuum filtration and the resin in column was washed with DMF six times.

Fmoc-4-Hyp(tBu)-OH (4.14 g; 36901, GL Biochem, Shanghai, China) and 2-(1H-benzotriazole-1-yl)-1,1,3,3-tetramethylaminium tetrafluoroborate (TBTU, 2.89 g; 00705, GL BiochemShanghai, China) were added to the resin. DMF (150 mL) was added to the reaction column, followed by N,N-diisopropylethylamine (DIPEA, 2.33 g; Suzhou Highfine Biotech Co. Ltd, Jiangsu, China). A Kaiser Test was carried out with few of the resin after 30 minutes reaction, a yellow colour of the solution and colourless gel indicated that the reaction was complete. The solvent was removed by vacuum filtration.

The above coupling step was repeated to couple the remaining amino acids in the same amounts (by mols): Fmoc-Tyr(tBu)-OH, Fmoc-Thr(tBu)-OH, Fmoc-4-Hyp(tBu)-OH, Fmoc-Tyr(tBu)-OH, Fmoc-Ser(tBu)-OH, Fmoc-Pro-OH, Fmoc-Lys(Boc)-OH and Boc-Ala-OH.

After Boc-Ala-OH was coupled on the resin, the resin was washed three times each with the following solvents, DMF (200 mL each time), DCM (200 mL each time) and methanol (200 mL each time). Then, the resin was dried by vacuum for about 2 hours.

120.0 mL (i.e. 10 mL per gram of the dried resin) of lysate, which comprised of 2% trifluoroacetic acid (TFA) in DCM, was added to immerse the resin-bounded peptide-containing compound. About 2 hours after cleavage, the solid support was removed by filtration and the filtrate was collected under reduced pressure. Then, the filtrate was concentrated by rotary distillation under reduced pressure.

After all solvents were removed, DMF (100 mL) was added to the flask to dissolve the solid, and procaine (2.13 g, Macklin Biochemical Co. Ltd., Shanghai, China), benzotriazol-1-yl-oxytripyrrolidinophosphonium hexafluorophosphate (PyBop, 4.68 g, P816085, Macklin Biochemical Co. Ltd., Shanghai, China), 4-dimethylaminopyridine (DMAP, 1.10 g, D807273, Macklin Biochemical Co. Ltd., Shanghai, China), and DIPEA (2.33 g) were added to the reaction solution.

After 16 hours, the reaction was complete. Precipitation of the final solution was carried out by adding 1200 mL (i.e. 10 mL per ml of the final solution) of saturated citric acid (Aladdin, Shanghai, China) water solution and the sediment was collected by filtration.

The sediments was then added to 120 mL (i.e. 10 mL per gram of the solid) of lysate, which comprised 95% trifluoroacetic acid (TFA), 2.5% water and 2.5% triisopropylsilane (Tis), which dissolved the peptide-containing solid. The side chains were deprotected during cleavage. After about 2 hours, the solution was precipitated with 1200 mL (i.e. 10 mL per ml of the filtrate) of diethyl ether and sediment was collected by filtration. The sediment was dried under vacuum for about 2 hours, yielding 4.09 g of crude title compound.

Analysis showed a target peak that was eluted at 12.505 minutes with the expected molecular weight (MS: m/z 1401.6). The purity was 45.564%.

4.0 g of the crude product was then dissolved in 50 mL of pure water and purified using LC3000 semi-preparation equipment. The preparation column model was a Dubhe-C18 model (Hanbon Sci. & Tech. Co., Ltd., Jiangsu, China) (50*250 mm, 100A column; detection: UV at 220 nm). The appropriate gradient for elution was calculated from LCMS detection step (Solvent A: 0.1% TFA in MeCN, solvent B: 0.1% TFA in water, with a linear gradient from 5%-20% solvent A concentration over 30 minutes; flow rate 60.0 mL/min).

Fractions were collected and analyzed using a Shimadzu LC-20 HPLC system (column as above, except with a linear gradient from 5%-30% solvent A concentration over 25 minutes).

Fractions with a purity of 98% were then mixed together for an anion exchange step. This was achieved using a LC3000 semi-preparation equipment (preparation column model: Dubhe-C18 model (as above). The fractions were diluted one time with pure water and loaded to the column directly, after that the column was washed with 0.37% of ammonium acetate in pure water for about 20 minutes followed by pure water for another 20 minutes at the flow rate of 60 mL/min, then eluted with the following gradient (Solvent A: 0.1% HAc in MeCN, solvent B: 0.1% HAc in water, with a linear gradient from 5%-20% solvent A concentration over 30 minutes; flow rate 60.0 mL/min). Fractions were collected and analyzed using Shimadzu LC-20 HPLC system (column and conditions as above). Fractions with a purity of 98% were mixed and freeze-dried to give 1.5 g of the purified title compound.

### Example 2

### Dental Treatment

A 1 mg/mL solution of the title compound of Example 1 was prepared in normal saline.

The resultant solution was sprayed using a standard spray device onto the dental ulcer of the patient. Pain relief occurred within 1 minute. The duration of pain relief was about 3 hours.

1. A conjugate compound formed between an anaesthetic compound and a peptide component of the amino acid sequence:
(SEQ ID NO: 3)
W-Lys-X¹-Ser-U-X²-Y
wherein:
W is absent or represents a 1, 2 or 3 amino acid sequence, in which the amino acids are selected from one or more of the group Ser, Lys, Ala, DOPA and a 3,4-dihydrocinnamic acid (HCA) residue, provided that, when present, the HCA residue is located at the N-terminus of the peptide sequence;
X¹ represents Pro, Hyp or diHyp;
U represents Tyr, DOPA or a single bond;
X² represents Ser, Pro, Hyp or diHyp; and
Y represents a 1 to 5 amino acid sequence, in which the amino acids are selected from one or more of the group Lys, Ala, Pro, Hyp, diHyp, Thr, DOPA and Tyr,
as well as regioisomers, stereoisomers, and pharmaceutically- or cosmetically-acceptable salts of said conjugates.

2. A compound as claimed in claim 1, wherein the anaesthetic is a local anaesthetic, and comprises a free amino group in its chemical structure, which forms an amide linkage with a free carboxylic acid moiety in the peptide component of amino acid SEQ ID No: 3.

3. A compound as claimed in claim 2, wherein the local anaesthetic is selected from the group ambucaine, benzocaine, butacaine, chloroprocaine, dimethocaine, metabutoxycaine, orthocaine, propantheline, propoxycaine, risocaine and procaine.

4. A compound as claimed in any one of the preceding claims, wherein, in the peptide component, W represents a 1 or 2 amino acid sequence, in which the amino acids are selected from one or more of the group Lys, Ala, DOPA and a HCA residue.

5. A compound as claimed in claim 4, wherein W represents HCA, HCA-Ala-, Ala, Lys-Ala, DOPA or DOPA-Ala-.

6. A compound as claimed in any one of the preceding claims, wherein, in the peptide component, Y represents a 3, 4 or 5 amino acid sequence, in which the amino acids are selected from one or more of the group Pro, Lys, Ala, Hyp, Thr, DOPA and Tyr.

7. A compound as claimed in claim 6, wherein Y represents a 4 amino acid sequence selected from the group -Pro-Y¹-Y²-Lys-, -Hyp-Y¹-Y²-Lys- and -Thr-Y¹-Y²-Lys-, wherein Y¹ and Y² are each independently selected from the group Pro, Ala, Hyp, Thr, DOPA and Tyr.

8. A compound as claimed in claim 7, wherein the amino acid sequence defined by Y is selected from the group: -Pro-Thr-DOPA-Lys-, -Pro-Thr-Tyr-Lys-, -Thr-Tyr-Pro-Lys-, -Thr-DOPA-Pro-Lys-, -Hyp-Thr-Tyr-Lys-, -Hyp-Thr-DOPA-Lys-, -Hyp-Thr-Ala-Lys-, -Thr-Tyr-Hyp-Lys-, -Thr-DOPA-Hyp-Lys-, -Thr-Ala-Hyp-Lys-, -Hyp-Thr-, -Thr-Tyr-, -Pro-Thr-, -Thr-DOPA-, -Thr-Tyr-Lys-, -Tyr-Pro-Lys-, -DOPA-Pro-Lys-, -Hyp-Thr-Tyr-, -Hyp-Thr-Tyr-Hyp-Lys-, -Thr-Tyr-Hyp-Lys-DOPA- and -Hyp-Thr-DOPA-.

9. A compound as claimed in any one of claims 1 to 3, wherein the conjugate comprised a peptide component of the amino acid sequence:
(SEQ ID NO: 63)
K-W¹-Lys-X¹-Ser-U-X²-Y¹-I-J
wherein K represents an optional N-terminal HCA group;
W¹ may be absent or represent a 1 or 2 amino acid sequence, in which the amino acids are selected from one or more of the group Ser, Lys, Ala and DOPA;
Y¹ represents a single bond or a 1 to 3 (e.g. a 1 or 2) amino acid sequence, in which the amino acids are selected from one or more of the group Lys, Ala, Pro, Hyp, diHyp, Thr, DOPA and Tyr;
I represents Pro, Hyp, diHyp, Thr, DOPA or Tyr;
J represents Lys or is absent; and
X¹, U and X² are as defined in claim 1.

10. A compound as claimed in claim 9, wherein, in the peptide component, W¹ represents Ala or Ser, or is absent.

11. A compound as claimed in claim 9 or claim 10, wherein, in the peptide component, X² represents Pro, Hyp or diHyp.

12. A compound as claimed in any one of claims 9 to 11, wherein, in the peptide component, when K is not present, W¹ represents Ala or is absent and J represents Lys, then I represents Pro, Hyp, diHyp or Thr.

13. A compound as claimed in any one of claims 9 to 12, wherein, in the peptide component, Y¹ represents a 1, a 2 or a 3 amino acid sequence, in which the amino acids are selected from the group Pro, Hyp, Thr, DOPA and Tyr.

14. A compound as claimed in any one of claims 9 to 13, wherein, in the peptide component, J represents Lys or is absent.

15. A compound as claimed in any one of claims 9 to 14, wherein, in the peptide component, I represents DOPA, Tyr, Pro or Hyp.

16. A compound as claimed in claim 15, wherein I represents DOPA or Tyr, more preferably Pro or, especially, Hyp.

17. A compound as claimed in any one of claims 9 to 16, wherein, in the peptide component, the amino acids in the sequence defined by Yl are selected from Pro, DOPA, Hyp, Thr and Tyr.

18. A compound as claimed in any one of claims 9 to 17, wherein, in the peptide component, in the amino acid sequence defined by Y¹, the amino acid DOPA, Thr, Lys or Tyr is linked to I.

19. A compound as claimed in any one of claims 9 to 18, wherein, in the peptide component, in the amino acid sequence defined by Y¹, the amino acid Pro, Hyp or Thr is linked to X².

20. A compound as claimed in any one of claims 9 to 19, wherein, in the peptide component, the amino acid sequence defined by Y¹ is -Hyp-Thr-Tyr-, -Hyp-Thr-DOPA-, -Thr-DOPA-Lys-, -Thr-Tyr-Lys-, -Thr-Tyr-, -Thr-DOPA-, -Pro-Thr- or -Hyp-Thr-.

21. A compound as claimed in any one of claims 9 to 19, wherein, in the peptide component, K is absent.

22. A compound as claimed in claim 21, wherein W¹ is absent, Y¹ represents a single bond and J represents Lys.

23. A compound as claimed in any one of the preceding claims, wherein, in the peptide component, U represents Tyr or DOPA.

24. A compound as claimed in any one of the preceding claims, wherein, in the peptide component, X¹ represents Hyp or Pro.

25. A compound as claimed in any one of the preceding claims, wherein, in the peptide component, X² represents diHyp, or Hyp.

26. A compound as claimed in any one of the preceding claims, wherein the peptide component is of the amino acid sequence:
(SEQ ID No: 4)
Ala-Lys-Pro-Ser-Tyr-Hyp-Thr-Tyr-Hyp-Lys-DOPA;
(SEQ ID No: 5)
Ala-Lys-Pro-Ser-Tyr-Pro-Pro-Thr-DOPA-Lys;
(SEQ ID No: 6)
Ala-Lys-Pro-Ser-Tyr-Pro-Thr-Tyr-Pro-Lys;
(SEQ ID No: 7)
Ala-Lys-Pro-Ser-Tyr-Pro-Thr-DOPA-Pro-Lys;
(SEQ ID No: 8)
Ala-Lys-Pro-Ser-Tyr-Pro-Hyp-Thr-Tyr-Lys;
(SEQ ID No: 9)
Ala-Lys-Pro-Ser-Tyr-Pro-Hyp-Thr-DOPA-Lys;
(SEQ ID No: 10)
Ala-Lys-Pro-Ser-Tyr-Hyp-Pro-Thr-Tyr-Lys;
(SEQ ID No: 11)
Ala-Lys-Pro-Ser-Tyr-Hyp-Pro-Thr-DOPA-Lys;
(SEQ ID No: 12)
Ala-Lys-Pro-Ser-Tyr-Hyp-Thr-Tyr-Hyp-Lys;
(SEQ ID No: 13)
Ala-Lys-Pro-Ser-Tyr-Hyp-Thr-DOPA-Hyp-Lys;
(SEQ ID No: 14)
Ala-Lys-Pro-Ser-Tyr-Hyp-Hyp-Thr-Tyr-Lys-DOPA;
(SEQ ID No: 15)
Ala-Lys-Pro-Ser-Tyr-Hyp-Hyp-Thr-DOPA-Lys-DOPA;
(SEQ ID No: 16)
Ala-Lys-Pro-Ser-Tyr-Hyp-Thr-DOPA-Hyp-Lys-DOPA;
(SEQ ID No: 17)
Lys-Ala-Lys-Pro-Ser-Tyr-Hyp-Hyp-Thr-DOPA;
(SEQ ID No: 18)
Lys-Ala-Lys-Hyp-Ser-Tyr-Hyp-Hyp-Thr-DOPA;
(SEQ ID No: 19)
Lys-Ala-Lys-Hyp-Ser-Tyr-Hyp-Hyp-Thr-Tyr;
(SEQ ID No: 20)
Lys-Ala-Lys-Pro-Ser-Tyr-Hyp-Hyp-Thr-Tyr;
(SEQ ID No: 21)
DOPA-Lys-Pro-Ser-Tyr-Hyp-Thr-Ala-Hyp-Lys;
(SEQ ID No: 22)
DOPA-Lys-Pro-Ser-Tyr-Hyp-Hyp-Thr-Ala-Lys;
(SEQ ID No: 23)
DOPA-Ala-Lys-Pro-Ser-Tyr-Hyp-Thr-Tyr-Hyp-Lys;
(SEQ ID No: 24)
DOPA-Ala-Lys-Pro-Ser-Tyr-Hyp-Hyp-Thr-Tyr-Lys;
(SEQ ID No: 25)
DOPA-Ala-Lys-Pro-Ser-Tyr-Hyp-Thr-DOPA-Hyp-Lys;
(SEQ ID No: 26)
DOPA-Ala-Lys-Pro-Ser-Tyr-Hyp-Hyp-Thr-DOPA-Lys;
(SEQ ID No: 27)
HCA-Lys-Pro-Ser-Tyr-Hyp-Hyp-Thr-Ala-Lys;
(SEQ ID No: 28)
HCA-Ala-Lys-Pro-Ser-Tyr-Hyp-Thr-Tyr-Hyp-Lys;
(SEQ ID No: 29)
HCA-Ala-Lys-Pro-Ser-Tyr-Hyp-Thr-DOPA-Hyp-Lys;
(SEQ ID No: 30)
HCA-Ala-Lys-Pro-Ser-Tyr-Hyp-Hyp-Thr-Tyr-Lys;
(SEQ ID No: 31)
HCA-Ala-Lys-Pro-Ser-Tyr-Hyp-Hyp-Thr-DOPA-Lys;
(SEQ ID No: 32)
HCA-Lys-Pro-Ser-Tyr-Hyp-Thr-Ala-Hyp-Lys;
(SEQ ID No: 33)
Lys-Ala-Lys-Hyp-Ser-DOPA-Hyp-Hyp-Thr-DOPA;
(SEQ ID No: 34)
Lys-Ala-Lys-Pro-Ser-DOPA-Hyp-Hyp-Thr-DOPA;
(SEQ ID No: 35)
Ala-Lys-Pro-Ser-DOPA-Pro-Pro-Thr-Tyr-Lys;
(SEQ ID No: 36)
Ala-Lys-Pro-Ser-DOPA-Pro-Pro-Thr-DOPA-Lys;
(SEQ ID No: 37)
Ala-Lys-Pro-Ser-DOPA-Pro-Thr-Tyr-Pro-Lys;
(SEQ ID No: 38)
Ala-Lys-Pro-Ser-DOPA-Pro-Thr-DOPA-Pro-Lys;
(SEQ ID No: 39)
Ala-Lys-Pro-Ser-DOPA-Pro-Hyp-Thr-Tyr-Lys;
(SEQ ID No: 40)
Ala-Lys-Pro-Ser-DOPA-Pro-Hyp-Thr-DOPA-Lys;
(SEQ ID No: 41)
Ala-Lys-Pro-Ser-DOPA-Hyp-Pro-Thr-Tyr-Lys;
(SEQ ID No: 42)
Ala-Lys-Pro-Ser-DOPA-Hyp-Pro-Thr-DOPA-Lys;
(SEQ ID No: 43)
Ala-Lys-Pro-Ser-DOPA-Hyp-Thr-Tyr-Hyp-Lys;
(SEQ ID No: 44)
Ala-Lys-Pro-Ser-DOPA-Hyp-Thr-DOPA-Hyp-Lys;
(SEQ ID No: 45)
Lys-Ala-Lys-Pro-Ser-DOPA-Hyp-Hyp-Thr-Tyr;
(SEQ ID No: 46)
Lys-Ala-Lys-Hyp-Ser-DOPA-Hyp-Hyp-Thr-Tyr;
(SEQ ID No: 47)
Ala-Lys-Pro-Ser-DOPA-Hyp-Hyp-Thr-Tyr-Lys-DOPA;
(SEQ ID No: 48)
Ala-Lys-Pro-Ser-DOPA-Hyp-Hyp-Thr-DOPA-Lys-DOPA;
(SEQ ID No: 49)
Ala-Lys-Pro-Ser-DOPA-Hyp-Thr-Tyr-Hyp-Lys-DOPA;
(SEQ ID No: 50)
Ala-Lys-Pro-Ser-DOPA-Hyp-Thr-DOPA-Hyp-Lys-DOPA;
(SEQ ID No: 51)
DOPA-Lys-Pro-Ser-DOPA-Hyp-Thr-Ala-Hyp-Lys;
(SEQ ID No: 52)
DOPA-Lys-Pro-Ser-DOPA-Hyp-Hyp-Thr-Ala-Lys;
(SEQ ID No: 53)
DOPA-Ala-Lys-Pro-Ser-DOPA-Hyp-Thr-DOPA-Hyp-Lys;
(SEQ ID No: 54)
DOPA-Ala-Lys-Pro-Ser-DOPA-Hyp-Hyp-Thr-Tyr-Lys;
(SEQ ID No: 55)
DOPA-Ala-Lys-Pro-Ser-DOPA-Hyp-Hyp-Thr-DOPA-Lys;
(SEQ ID No: 56)
HCA-Ala-Lys-Pro-Ser-DOPA-Hyp-Thr-DOPA-Hyp-Lys;
(SEQ ID No: 57)
HCA-Ala-Lys-Pro-Ser-DOPA-Hyp-Hyp-Thr-DOPA-Lys;
(SEQ ID No: 58)
HCA-Lys-Pro-Ser-DOPA-Hyp-Thr-Ala-Hyp-Lys;
(SEQ ID No: 59)
HCA-Lys-Pro-Ser-DOPA-Hyp-Hyp-Thr-Ala-Lys;
(SEQ ID No: 60)
DOPA-Ala-Lys-Pro-Ser-DOPA-Hyp-Thr-Tyr-Hyp-Lys;
(SEQ ID No: 61)
HCA-Ala-Lys-Pro-Ser-DOPA-Hyp-Thr-Tyr-Hyp-Lys;
(SEQ ID No: 62).
HCA-Ala-Lys-Pro-Ser-DOPA-Hyp-Hyp-Thr-Tyr-Lys;
(SEQ ID No: 64)
Lys-Pro-Ser-Tyr-Hyp-Hyp-Thr-DOPA-Hyp-Lys;
(SEQ ID No: 65)
Lys-Pro-Ser-Tyr-Hyp-Hyp-Thr-Tyr-Hyp-Lys;
(SEQ ID No: 66)
Lys-Pro-Ser-Tyr-Hyp-Thr-DOPA-Hyp-Lys;
(SEQ ID No: 67)
Lys-Pro-Ser-Tyr-Hyp-Thr-Tyr-Hyp-Lys;
(SEQ ID No: 68)
Ser-Lys-Pro-Ser-Tyr-Hyp-Hyp-Thr-DOPA-Lys;
(SEQ ID No: 69)
Ser-Lys-Pro-Ser-Tyr-Hyp-Hyp-Thr-Tyr-Lys;
(SEQ ID No: 70)
Ser-Lys-Pro-Ser-Tyr-Hyp-Thr-DOPA-Hyp-Lys;
(SEQ ID No: 71)
Ser-Lys-Pro-Ser-Tyr-Hyp-Thr-Tyr-Hyp-Lys;
(SEQ ID No: 72)
Ala-Lys-Pro-Ser-Tyr-Hyp-Hyp-Thr-DOPA-Hyp-Lys;
(SEQ ID No: 73)
Ala-Lys-Pro-Ser-Tyr-Hyp-Hyp-Thr-Tyr-Hyp-Lys;
(SEQ ID No: 74)
Ala-Lys-Pro-Ser-Tyr-Hyp-Thr-DOPA-Hyp-Lys;
(SEQ ID No: 75)
Ala-Lys-Pro-Ser-Tyr-Hyp-Thr-Tyr-Hyp-Lys;
(SEQ ID No: 76)
Lys-Pro-Ser-Tyr-Hyp-Hyp-Thr-DOPA-Hyp;
(SEQ ID No: 77)
Lys-Pro-Ser-Tyr-Hyp-Hyp-Thr-Tyr-Hyp;
(SEQ ID No: 78)
Lys-Pro-Ser-Tyr-Hyp-Thr-DOPA-Hyp;
(SEQ ID No: 79)
Lys-Pro-Ser-Tyr-Hyp-Thr-Tyr-Hyp;
(SEQ ID No: 80)
Ser-Lys-Pro-Ser-Tyr-Hyp-Hyp-Thr-Tyr;
(SEQ ID No: 81)
Ser-Lys-Pro-Ser-Tyr-Hyp-Thr-DOPA-Hyp;
(SEQ ID No: 82)
Ser-Lys-Pro-Ser-Tyr-Hyp-Thr-Tyr-Hyp;
(SEQ ID No: 83)
Ala-Lys-Pro-Ser-Tyr-Hyp-Hyp-Thr-DOPA-Hyp;
(SEQ ID No: 84)
Ala-Lys-Pro-Ser-Tyr-Hyp-Hyp-Thr-Tyr-Hyp;
(SEQ ID No: 85)
Ala-Lys-Pro-Ser-Tyr-Hyp-Thr-DOPA-Hyp;
(SEQ ID No: 86)
Ala-Lys-Pro-Ser-Tyr-Hyp-Thr-Tyr-Hyp;
(SEQ ID No: 87)
Ser-Lys-Pro-Ser-Tyr-Hyp-Hyp-Thr-DOPA;
(SEQ ID No: 88)
HCA-Ala-Lys-Pro-Ser-Tyr-Hyp-Hyp-Thr-Tyr-Lys;
(SEQ ID No: 89)
HCA-Ala-Lys-Pro-Ser-Tyr-Hyp-Thr-Tyr-Hyp-Lys;
(SEQ ID No: 90)
Ala-Lys-Pro-Ser-DOPA-Hyp-Thr-DOPA-Hyp-Lys;
(SEQ ID No: 91)
Ala-Lys-Pro-Ser-Pro-Thr-Tyr-Pro-Lys;
(SEQ ID No: 92)
Ala-Lys-Hyp-Ser-Tyr-Hyp-Thr-Tyr-Hyp-Lys;
(SEQ ID No: 93)
Ala-Lys-Hyp-Ser-DOPA-Hyp -Thr-DOPA-Hyp-Lys;
(SEQ ID No: 94)
Ala-Lys-Hyp-Ser-Tyr-Hyp-Thr-DOPA-Hyp-Lys;
(SEQ ID No: 95)
HCA-Ala-Lys-Pro-Ser-Tyr-Hyp-Hyp-Thr-Tyr;
(SEQ ID No: 96)
HCA-Ala-Lys-Pro-Ser-Tyr-Hyp-Thr-Tyr-Hyp;
(SEQ ID No: 97)
Ala-Lys-Pro-Ser-DOPA-Hyp-Thr-DOPA-Hyp;
(SEQ ID No: 98)
Ala-Lys-Pro-Ser-Pro-Thr-Tyr-Pro;
(SEQ ID No: 99)
Ala-Lys-Hyp-Ser-Tyr-Hyp-Thr-Tyr-Hyp;
(SEQ ID No: 100)
Ala-Lys-Hyp-Ser-DOPA-Hyp -Thr-DOPA-Hyp;
(SEQ ID No: 101)
Ala-Lys-Hyp-Ser-Tyr-Hyp-Thr-DOPA-Hyp;
(SEQ ID No: 102)
Ala-Lys-Pro-Ser-DOPA-Hyp-Thr-Tyr-Lys-Hyp;
(SEQ ID No: 103)
Ala-Lys-Hyp-Ser-DOPA-Hyp-Thr-Tyr-Lys-Hyp;
(SEQ ID No: 104)
Ala-Lys-Pro-Ser-Tyr-Hyp-Thr-Tyr-Lys-Hyp;
(SEQ ID No: 105)
Ala-Lys-Hyp-Ser-Tyr-Hyp-Thr-Tyr-Lys-Hyp;
(SEQ ID No: 106)
Ala-Lys-Pro-Ser-Tyr-Hyp-Thr-DOPA-Lys-Hyp;
(SEQ ID No: 107)
Ala-Lys-Hyp-Ser-Tyr-Hyp-Thr-DOPA-Lys-Hyp;
(SEQ ID No: 108)
Ala-Lys-Pro-Ser-DOPA-Hyp-Thr-DOPA-Lys-Hyp;
(SEQ ID No: 109)
Ala-Lys-Hyp-Ser-DOPA-Hyp-Thr-DOPA-Lys-Hyp;
(SEQ ID No: 111)
Lys-Pro-Ser-Tyr-Hyp-DOPA-Lys;
or
(SEQ ID No: 112)
Lys-Pro-Ser-Tyr-Hyp-Tyr-Lys.

27. A compound as claimed in claim 25, wherein the peptide component is of the amino acid sequence:
(SEQ ID No: 1)
Ala-Lys-Pro-Ser-Tyr-Hyp-Hyp-Thr-DOPA-Lys;
(SEQ ID No: 2)
Ala-Lys-Pro-Ser-Tyr-Hyp-Hyp-Thr-Tyr-Lys;
(SEQ ID No: 12)
Ala-Lys-Pro-Ser-Tyr-Hyp-Thr-Tyr-Hyp-Lys;
(SEQ ID No: 13)
Ala-Lys-Pro-Ser-Tyr-Hyp-Thr-DOPA-Hyp-Lys;
(SEQ ID No: 104)
Ala-Lys-Pro-Ser-Tyr-Hyp-Thr-Tyr-Lys-Hyp;
(SEQ ID No: 111)
Lys-Pro-Ser-Tyr-Hyp-DOPA-Lys;
or
(SEQ ID NO: 112)
Lys-Pro-Ser-Tyr-Hyp-Tyr-Lys.

28. A compound as defined in any one of the preceding claims, for use in human or animal medicine.

29. A compound as defined in any one of claims 1 to 27, for use as a pharmaceutical.

30. A pharmaceutical formulation comprising a compound as defined in any one of claims 1 to 27 and a pharmaceutically-acceptable, adjuvant, diluent or carrier.

31. A pharmaceutical formulation as claimed in claim 30 that is suitable for, adapted for, and/or packaged and presented for local delivery by topical administration and/or or by injection.

32. A pharmaceutical formulation as claimed in claim 30 that is suitable for, adapted for, and/or packaged and presented for systemic delivery by injection.

33. A pharmaceutical formulation as claimed in any one of claims 30 to 32, which further includes a further, pharmaceutically-active ingredient.

34. A kit of parts comprising components:
(A) a compound as defined in any one of claims 1 to 27, or a pharmaceutical formulation as defined in any one of claims 30 to 32; and
(B) a pharmaceutical formulation including a further pharmaceutically-active ingredient in admixture with a pharmaceutically-acceptable adjuvant, diluent or carrier, which components (A) and (B) are each provided in a form that is suitable for administration in conjunction with the other.

35. A pharmaceutical formulation as claimed in claim 33, or a kit of parts as claimed in claim 34 wherein the pharmaceutically-active ingredient is an anti-inflammatory agent.

36. A compound as defined in any one of claims 1 to 27, a formulation as claimed in any one of claim 30 to 33 or 35, or a kit of parts as claimed in claim 34 or claim 35, for use in the treatment of inflammation, an inflammatory disorder, and/or of a disorder characterised by inflammation and/or in the treatment of pain.

37. The use of a compound as defined in any one of claims 1 to 27, a formulation as claimed in any one of claim 30 to 33 or 35, or a kit of parts as claimed in claim 34 or claim 35, for the manufacture a medicament for the treatment of inflammation, an inflammatory disorder, and/or of a disorder characterised by inflammation and/or in the treatment of pain.

38. A method of treatment of inflammation, an inflammatory disorder, and/or of a disorder characterised by inflammation and/or in the treatment of pain, which method comprises the administration of a compound as defined in any one of claims 1 to 27, a formulation as claimed in any one of claim 30 to 33 or 35, or a kit of parts as claimed in claim 34 or claim 35, to a patient in need of such treatment.

39. A compound, a formulation or a kit of parts for use as claimed in claim 36, a use as claimed in claim 37, or a method as claimed in claim 38, wherein the disorder characterised by inflammation is, or results in, a wound or a burn.

40. A compound, a formulation or a kit of parts for use as claimed in claim 36, a use as claimed in claim 37, or a method as claimed in claim 38, wherein treatment relieves pain and/or results in anaesthesia.

41. A compound, a formulation or a kit of parts for use, a use, or a method as claimed in claim 40, wherein the treatment is of acute pain by transdermal, by intradermal, by transmucosal, subcutaneous and/or by intramucosal administration, by infiltration, by brachial plexus block, by epidural (extradural) block, by spinal anesthesia (subarachnoid block) and/or by iontophoresis, achieved by injection on a local basis or by other form of local and/or topical application, prior to, during and/or after a surgical or a diagnostic procedure.

42. A compound, a formulation or a kit of parts for use, a use, or a method as claimed in claim 41, wherein the surgical and diagnostic procedures comprises one of more of the group consisting of a surgical intervention, a diagnostic intervention, a dental procedure, skin surgery, laser surgery to treat melanin pigmentation, a cosmetic procedure, peripherial blood vessel surgery, podiatry, surgery to mucosal surfaces, eye surgery, ear nose and throat operations, shoulder and/or arm surgery, surgery to a joint in the human body; surgery to one or more internal organs, drainage of bodily fluids, insertion of a medical device, venipuncture, intravenous cannula insertion, bone joint surgery, a spinal procedure, a gynaecological procedure, a urological procedure, a gastrointestinal endoscopy, a colonoscopy, a brochioscopy, intubation and an intervention in an obstetric procedure and/or childbirth.

43. A compound, a formulation or a kit of parts for use, a use, or a method as claimed in claim 40, wherein the pain relief is part of the treatment of a disease and/or a condition.

44. A compound, a formulation or a kit of parts for use, a use, or a method as claimed in claim 43, wherein the disease or condition, is selected from stomatitis, oral mucositis, Burning Mouth Syndrome, Sjögren's syndrome, xerostomia, periodontitis, toothache, a throat infection, pharyngitis; a canker sore, an aphthous ulcer. any break in the mucous membrane, proctitis and colitis.


SEQUENCE LISTING (128 sequences)

SEQ ID NO: 1 - protein, 10 aa, Unknown
  Mefp-1 decapeptide
  SITE 6,7: Wherein Xaa represents hydroxyproline
  SITE 9: Wherein Xaa is 3,4-dihydroxyphenylalanine
  SITE 10: Wherein Lys is optionally C-terminally modified with a local anaesthetic selected from Procaine, Tetracaine, Dimethocaine, Benzocaine, Orthocaine, and Risocaine, or is not so modified
AKPSYXXTXK

SEQ ID NO: 2 - protein, 10 aa, Unknown
  Mefp-1 decapeptide analogue
  SITE 6,7: Wherein Xaa is hydroxyproline
  SITE 10: Wherein Lys is optionally C-terminally modified with a local anaesthetic selected from Procaine, Tetracaine, Dimethocaine, Benzocaine, Orthocaine, and Risocaine, or is not so modified
AKPSYXXTYK

SEQ ID NO: 3 - protein, 13 aa, Artificial Sequence
  Peptide component
  VARIANT 1,2,3: Wherein Xaa is absent or is are selected from one or more of the group serine, lysine, alanine, and 3,4-dihydroxyphenylalanine
  VARIANT 2: Wherein where Xaa at position 1 is absent, Xaa is optionally N-terminally modified with a 3,4-diydrocinnamic acid group, or is not so modified
  VARIANT 3: Wherein where Xaa at positions 1 and 2 are absent, Xaa is optionally N-terminally modified with a 3,4-diydrocinnamic acid group, or is not so modified
  VARIANT 4: Wherein where Xaa at positions 1, 2, and 3 is absent, Lys is optionally N-terminally modified with a 3,4-diydrocinnamic acid group, or is not so modified
  VARIANT 5: Wherein Xaa is selected from proline, hydroxyproline, or dihydroxyproline
  VARIANT 7: Wherein Xaa is absent or is selected from tyrosine or 3,4-dihydroxyphenylalanine
  VARIANT 8: Wherein Xaa is selected from serine, proline, hydroxyproline, or dihydroxyproline
  VARIANT 9: Wherein Xaa is selected from the group lysine, alanine, proline, hydroxyproline, dihydroxyproline, threonine, 3,4-dihydroxyphenylalanine, and tyrosine
  VARIANT 10..13: Wherein Xaa is absent or is selected from the group lysine, alanine, proline, hydroxyproline, dihydroxyproline, threonine, 3,4-dihydroxyphenylalanine, and tyrosine
XXXKXSXXXXXXX

SEQ ID NO: 4 - protein, 11 aa, Artificial Sequence
  Peptide component
  SITE 6,9: Wherein Xaa is hydroxyproline
  SITE 11: Wherein Xaa is 3,4-dihydroxyphenylalanine
AKPSYXTYXKX

SEQ ID NO: 5 - protein, 10 aa, Artificial Sequence
  Peptide component
  SITE 9: Wherein Xaa is 3,4-dihydroxyphenylalanine
AKPSYPPTXK

SEQ ID NO: 6 - protein, 10 aa, Artificial Sequence
  Peptide component
AKPSYPTYPK

SEQ ID NO: 7 - protein, 10 aa, Artificial Sequence
  Peptide component
  SITE 8: Wherein Xaa is 3,4-dihydroxyphenylalanine
AKPSYPTXPK

SEQ ID NO: 8 - protein, 10 aa, Artificial Sequence
  Peptide component
  SITE 7: Wherein Xaa is hydroxyproline
AKPSYPXTYK

SEQ ID NO: 9 - protein, 10 aa, Artificial Sequence
  Peptide component
  SITE 7: Wherein Xaa is hydroxyproline
  SITE 9: Wherein Xaa is 3,4-dihydroxyphenylalanine
AKPSYPXTXK

SEQ ID NO: 10 - protein, 10 aa, Artificial Sequence
  Peptide component
  SITE 6: Wherein Xaa is dihydroxyproline
AKPSYXPTYK

SEQ ID NO: 11 - protein, 10 aa, Artificial Sequence
  Peptide component
  SITE 6: Wherein Xaa is hydroxyproline
  SITE 9: Wherein Xaa is 3,4-dihydroxyphenylalanine
AKPSYXPTXK

SEQ ID NO: 12 - protein, 10 aa, Artificial Sequence
  Peptide component
  SITE 6,9: Wherein Xaa is hydroxyproline
  SITE 10: Wherein Lys is optionally C-terminally modified with a local anaesthetic selected from Procaine, Tetracain, Dimethocaine, Benzocaine, Orthocaine, Butacaine, Ambucaine, Chloroprocaine, Metabutoxycaine, Propantheline, Risocaine, and Propoxycaine, or is not so modified
AKPSYXTYXK

SEQ ID NO: 13 - protein, 10 aa, Artificial Sequence
  Peptide component
  SITE 6,9: Wherein Xaa is hydroxyproline
  SITE 8: Wherein Xaa is 3,4-dihydroxyphenylalanine
  SITE 10: Wherein Lys is optionally C-terminally modified with a local anaesthetic selected from Procaine, Tetracaine, Dimethocaine, Benzocaine, Orthocaine, and Risocaine, or is not so modified
AKPSYXTXXK

SEQ ID NO: 14 - protein, 11 aa, Artificial Sequence
  Peptide component
  SITE 6,7: Wherein Xaa is hydroxyproline
  SITE 11: Wherein Xaa is 3,4-dihydroxyphenylalanine
AKPSYXXTYKX

SEQ ID NO: 15 - protein, 11 aa, Artificial Sequence
  Peptide component
  SITE 6,7: Wherein Xaa is hydroxyproline
  SITE 9,11: Wherein Xaa is 3,4-dihydroxyphenylalanine
AKPSYXXTXKX

SEQ ID NO: 16 - protein, 11 aa, Artificial Sequence
  Peptide component
  SITE 6,9: Wherein Xaa is hydroxyproline
  SITE 8,11: Wherein Xaa is 3,4-dihydroxyphenylalanine
AKPSYXTXXKX

SEQ ID NO: 17 - protein, 10 aa, Artificial Sequence
  Peptide component
  SITE 7,8: Wherein Xaa is hydroxyproline
  SITE 10: Wherein Xaa is 3,4-dihydroxyphenylalanine
KAKPSYXXTX

SEQ ID NO: 18 - protein, 10 aa, Artificial Sequence
  Peptide component
  SITE 4,7,8: Wherein Xaa is hydroxyproline
  SITE 10: Wherein Xaa is 3,4-dihydroxyphenylalanine
KAKXSYXXTX

SEQ ID NO: 19 - protein, 10 aa, Artificial Sequence
  Peptide component
  SITE 4,7,8: Wherein Xaa is hydroxyproline
KAKXSYXXTY

SEQ ID NO: 20 - protein, 10 aa, Artificial Sequence
  Peptide component
  SITE 7,8: Wherein Xaa is hydroxyproline
KAKPSYXXTY

SEQ ID NO: 21 - protein, 10 aa, Artificial Sequence
  SITE 1: Wherein Xaa is 3,4-dihydroxyphenylalanine
  Peptide component
  SITE 6,9: Wherein Xaa is hydroxyproline
XKPSYXTAXK

SEQ ID NO: 22 - protein, 10 aa, Artificial Sequence
  SITE 1: Wherein Xaa is 3,4-dihydroxyphenylalanine
  Peptide component
  SITE 6,7: Wherein Xaa is hydroxyproline
XKPSYXXTAK

SEQ ID NO: 23 - protein, 11 aa, Artificial Sequence
  SITE 1: Wherein Xaa is 3,4-dihydroxyphenylalanine
  Peptide component
  SITE 7,10: Wherein Xaa is hydroxyproline
XAKPSYXTYXK

SEQ ID NO: 24 - protein, 11 aa, Artificial Sequence
  SITE 1: Wherein Xaa is 3,4-dihydroxyphenylalanine
  Peptide component
  SITE 7,8: Wherein Xaa is hydroxyproline
XAKPSYXXTYK

SEQ ID NO: 25 - protein, 11 aa, Artificial Sequence
  SITE 1,9: Wherein Xaa is 3,4-dihydroxyphenylalanine
  Peptide component
  SITE 7,10: Wherein Xaa is hydroxyproline
XAKPSYXTXXK

SEQ ID NO: 26 - protein, 11 aa, Artificial Sequence
  SITE 1,10: Wherein Xaa is 3,4-dihydroxyphenylalanine
  Peptide component
  SITE 7,8: Wherein Xaa is hydroxyproline
XAKPSYXXTXK

SEQ ID NO: 27 - protein, 9 aa, Artificial Sequence
  SITE 1: Wherein Lys is N-terminally modified with 3,4-Dihydrocinnamic acid
  Peptide component
  SITE 5,6: Wherein Xaa is hydroxyproline
KPSYXXTAK

SEQ ID NO: 28 - protein, 10 aa, Artificial Sequence
  SITE 1: Wherein Ala is N-terminally modified with 3,4-Dihydrocinnamic acid
  Peptide component
  SITE 6,9: Wherein Xaa is hydroxyproline
AKPSYXTYXK

SEQ ID NO: 29 - protein, 10 aa, Artificial Sequence
  SITE 1: Wherein Ala is N-terminally modified with a 3,4-Dihydrocinnamic acid group
  Peptide component
  SITE 6,9: Wherein Xaa is hydroxyproline
  SITE 8: Wherein Xaa is 3,4-dihydroxyphenylalanine
AKPSYXTXXK

SEQ ID NO: 30 - protein, 10 aa, Artificial Sequence
  SITE 1: Wherein Ala is N-terminally modified with a 3,4-Dihydrocinnamic acid group
  Peptide component
  SITE 6,7: Wherein Xaa is hydroxyproline
AKPSYXXTYK

SEQ ID NO: 31 - protein, 10 aa, Artificial Sequence
  SITE 1: Wherein Ala is N-terminally modified with a 3,4-Dihydrocinnamic acid group
  Peptide component
  SITE 6,7: Wherein Xaa is hydroxyproline
  SITE 9: Wherein Xaa is 3,4-dihydroxyphenylalanine
AKPSYXXTXK

SEQ ID NO: 32 - protein, 9 aa, Artificial Sequence
  SITE 1: Wherein Lys is N-terminally modified with a 3,4-Dihydrocinnamic acid group
  Peptide component
  SITE 5,8: Wherein Xaa is hydroxyproline
KPSYXTAXK

SEQ ID NO: 33 - protein, 10 aa, Artificial Sequence
  Peptide component
  SITE 4,7,8: Wherein Xaa is hydroxyproline
  SITE 6,10: Wherein Xaa is 3,4-dihydroxyphenylalanine
KAKXSXXXTX

SEQ ID NO: 34 - protein, 10 aa, Artificial Sequence
  Peptide component
  SITE 6,10: Wherein Xaa is 3,4-dihydroxyphenylalanine
  SITE 7,8: Wherein Xaa is hydroxyproline
KAKPSXXXTX

SEQ ID NO: 35 - protein, 10 aa, Artificial Sequence
  Peptide component
  SITE 5: Wherein Xaa is 3,4-dihydroxyphenylalanine
AKPSXPPTYK

SEQ ID NO: 36 - protein, 10 aa, Artificial Sequence
  Peptide component
  SITE 5,9: Wherein Xaa is 3,4-dihydroxyphenylalanine
AKPSXPPTXK

SEQ ID NO: 37 - protein, 10 aa, Artificial Sequence
  Peptide component
  SITE 5: Wherein Xaa is 3,4-dihydroxyphenylalanine
AKPSXPTYPK

SEQ ID NO: 38 - protein, 10 aa, Artificial Sequence
  Peptide component
  SITE 5,8: Wherein Xaa is 3,4-dihydroxyphenylalanine
AKPSXPTXPK

SEQ ID NO: 39 - protein, 10 aa, Artificial Sequence
  Peptide component
  SITE 5: Wherein Xaa is 3,4-dihydroxyphenylalanine
  SITE 7: Wherein Xaa is hydroxyproline
AKPSXPXTYK

SEQ ID NO: 40 - protein, 10 aa, Artificial Sequence
  Peptide component
  SITE 5,9: Wherein Xaa is 3,4-dihydroxyphenylalanine
  SITE 7: Wherein Xaa is hydroxyproline
AKPSXPXTXK

SEQ ID NO: 41 - protein, 10 aa, Artificial Sequence
  Peptide component
  SITE 5: Wherein Xaa is 3,4-dihydroxyphenylalanine
  SITE 6: Wherein Xaa is hydroxyproline
AKPSXXPTYK

SEQ ID NO: 42 - protein, 10 aa, Artificial Sequence
  Peptide component
  SITE 5,9: Wherein Xaa is 3,4-dihydroxyphenylalanine
  SITE 6: Wherein Xaa is hydroxyproline
AKPSXXPTXK

SEQ ID NO: 43 - protein, 10 aa, Artificial Sequence
  Peptide component
  SITE 5: Wherein Xaa is 3,4-dihydroxyphenylalanine
  SITE 6,9: Wherein Xaa is hydroxyproline
AKPSXXTYXK

SEQ ID NO: 44 - protein, 10 aa, Artificial Sequence
  Peptide component
  SITE 5,8: Wherein Xaa is 3,4-dihydroxyphenylalanine
  SITE 6,9: Wherein Xaa is hydroxyproline
AKPSXXTXXK

SEQ ID NO: 45 - protein, 10 aa, Artificial Sequence
  Peptide component
  SITE 6: Wherein Xaa is 3,4-dihydroxyphenylalanine
  SITE 7,8: Wherein Xaa is hydroxyproline
KAKPSXXXTY

SEQ ID NO: 46 - protein, 10 aa, Artificial Sequence
  Peptide component
  SITE 4,7,8: Wherein Xaa is hydroxyproline
  SITE 6: Wherein Xaa is 3,4-dihydroxyphenylalanine
KAKXSXXXTY

SEQ ID NO: 47 - protein, 11 aa, Artificial Sequence
  Peptide component
  SITE 5,11: Wherein Xaa is 3,4-dihydroxyphenylalanine
  SITE 6,7: Wherein Xaa is hydroxyproline
AKPSXXXTYKX

SEQ ID NO: 48 - protein, 11 aa, Artificial Sequence
  Peptide component
  SITE 5,9,11: Wherein Xaa is 3,4-dihydroxyphenylalanine
  SITE 6,7: Wherein Xaa is hydroxyproline
AKPSXXXTXKX

SEQ ID NO: 49 - protein, 11 aa, Artificial Sequence
  Peptide component
  SITE 5,11: Wherein Xaa is 3,4-dihydroxyphenylalanine
  SITE 6,9: Wherein Xaa is hydroxyproline
AKPSXXTYXKX

SEQ ID NO: 50 - protein, 11 aa, Artificial Sequence
  Peptide component
  SITE 5,8,11: Wherein Xaa is 3,4-dihydroxyphenylalanine
  SITE 6,9: Wherein Xaa is hydroxyproline
AKPSXXTXXKX

SEQ ID NO: 51 - protein, 10 aa, Artificial Sequence
  SITE 1,5: Wherein Xaa is 3,4-dihydroxyphenylalanine
  Peptide component
  SITE 6,9: Wherein Xaa is hydroxyproline
XKPSXXTAXK

SEQ ID NO: 52 - protein, 10 aa, Artificial Sequence
  SITE 1,5: Wherein Xaa is 3,4-dihydroxyphenylalanine
  Peptide component
  SITE 6,7: Wherein Xaa is hydroxyproline
XKPSXXXTAK

SEQ ID NO: 53 - protein, 11 aa, Artificial Sequence
  SITE 1,6,9: Wherein Xaa is 3,4-dihydroxyphenylalanine
  Peptide component
  SITE 7,10: Wherein Xaa is hydroxyproline
XAKPSXXTXXK

SEQ ID NO: 54 - protein, 11 aa, Artificial Sequence
  SITE 1,6: Wherein Xaa is 3,4-dihydroxyphenylalanine
  Peptide component
  SITE 7,8: Wherein Xaa is hydroxyproline
XAKPSXXXTYK

SEQ ID NO: 55 - protein, 11 aa, Artificial Sequence
  SITE 1,6,10: Wherein Xaa is 3,4-dihydroxyphenylalanine
  Peptide component
  SITE 7,8: Wherein Xaa is hydroxyproline
XAKPSXXXTXK

SEQ ID NO: 56 - protein, 10 aa, Artificial Sequence
  SITE 1: Wherein Ala is N-terminally modified with a 3,4-Dihydrocinnamic acid group
  Peptide component
  SITE 5,8: Wherein Xaa is 3,4-dihydroxyphenylalanine
  SITE 6,9: Wherein Xaa is hydroxyproline
AKPSXXTXXK

SEQ ID NO: 57 - protein, 10 aa, Artificial Sequence
  SITE 1: Wherein Ala is N-terminally modified with a 3,4-Dihydrocinnamic acid group
  Peptide component
  SITE 5,9: Wherein Xaa is 3,4-dihydroxyphenylalanine
  SITE 6,7: Wherein Xaa is hydroxyproline
AKPSXXXTXK

SEQ ID NO: 58 - protein, 9 aa, Artificial Sequence
  SITE 1: Wherein Lys is N-terminally modified with a 3,4-Dihydrocinnamic acid group
  Peptide component
  SITE 4: Wherein Xaa is 3,4-dihydroxyphenylalanine
  SITE 5,8: Wherein Xaa is hydroxyproline
KPSXXTAXK

SEQ ID NO: 59 - protein, 9 aa, Artificial Sequence
  SITE 1: Wherein Lys is N-terminally modified with a 3,4-Dihydrocinnamic acid group
  Peptide component
  SITE 4: Wherein Xaa is 3,4-dihydroxyphenylalanine
  SITE 5,6: Wherein Xaa is hydroxyproline
KPSXXXTAK

SEQ ID NO: 60 - protein, 11 aa, Artificial Sequence
  SITE 1,6: Wherein Xaa is 3,4-dihydroxyphenylalanine
  Peptide component
  SITE 7,10: Wherein Xaa is hydroxyproline
XAKPSXXTYXK

SEQ ID NO: 61 - protein, 10 aa, Artificial Sequence
  SITE 1: Wherein Ala is N-terminally modified with a 3,4-Dihydrocinnamic acid group
  Peptide component
  SITE 5: Wherein Xaa is 3,4-dihydroxyphenylalanine
  SITE 6,9: Wherein Xaa is hydroxyproline
AKPSXXTYXK

SEQ ID NO: 62 - protein, 10 aa, Artificial Sequence
  SITE 1: Wherein Ala is N-terminally modified with a 3,4-Dihydrocinnamic acid group
  Peptide component
  SITE 5: Wherein Xaa is 3,4-dihydroxyphenylalanine
  SITE 6,7: Wherein Xaa is hydroxyproline
AKPSXXXTYK

SEQ ID NO: 63 - protein, 12 aa, Artificial Sequence
  SITE 1: Wherein Xaa is optionally N-terminally modified with a 3,4-Dihydrocinnamic acid group
  Peptide component
  VARIANT 1,2: Wherein Xaa is absent or is selcted from serine, lysine, alanine, or 3,4-dihydroxyphenylalanine
  VARIANT 4: Wherein Xaa is selected from proline, hydroxyproline, or dihydroxyproline
  VARIANT 6: Wherein Xaa is absent or is selected from tyrosine or 3,4-dihydroxyphenylalanine
  VARIANT 7: Wherein Xaa is serine, proline, hydroxyproline, or dihydroxyproline
  VARIANT 8,9,10: Wherein Xaa is absent or is selected from the group lysine, alanine, proline, hydroxyproline, dihydroxyproline, threonine, 3,4-dihydroxyphenylalanine, and tyrosine
  VARIANT 11: Wherein Xaa is selected from proline, hydroxyproline, dihydroxyproline, threonine, 3,4-dihydroxyphenylalanine, and tyrosine
  VARIANT 12: Wherein Xaa is absent or is lysine
XXKXSXXXXXXX

SEQ ID NO: 64 - protein, 10 aa, Artificial Sequence
  Peptide component
  SITE 5,6,9: Wherein Xaa is hydroxyproline
  SITE 8: Wherein Xaa is 3,4-dihydroxyphenylalanine
  SITE 10: Wherein Lys is optionally C-terminally modified with a local anaesthetic selected from Procaine, Tetracaine, Dimethocaine, Benzocaine, Orthocaine, and Risocaine, or is not so modified
KPSYXXTXXK

SEQ ID NO: 65 - protein, 10 aa, Artificial Sequence
  Peptide component
  SITE 5,6,9: Wherein Xaa is hydroxyproline
  SITE 10: Wherein Lys is optionally C-terminally modified with a local anaesthetic selected from Procaine, Tetracaine, Dimethocaine, Benzocaine, Orthocaine, and Risocaine, or is not so modified
KPSYXXTYXK

SEQ ID NO: 66 - protein, 9 aa, Artificial Sequence
  Peptide component
  SITE 5,8: Wherein Xaa is hydroxyproline
  SITE 7: Wherein Xaa is 3,4-dihydroxyphenylalanine
  SITE 9: Wherein Lys is optionally C-terminally modified with a local anaesthetic selected from Procaine, Tetracaine, Dimethocaine, Benzocaine, Orthocaine, and Risocaine, or is not so modified
KPSYXTXXK

SEQ ID NO: 67 - protein, 9 aa, Artificial Sequence
  Peptide component
  SITE 5,8: Wherein Xaa is hydroxyproline
  SITE 9: Wherein Lys is optionally C-terminally modified with a local anaesthetic selected from Procaine, Tetracaine, Dimethocaine, Benzocaine, Orthocaine, and Risocaine, or is not so modified
KPSYXTYXK

SEQ ID NO: 68 - protein, 10 aa, Artificial Sequence
  Peptide component
  SITE 6,7: Wherein Xaa is hydroxyproline
  SITE 9: Wherein Xaa is 3,4-dihydroxyphenylalanine
  SITE 10: Wherein Lys is optionally C-terminally modified with a local anaesthetic selected from Procaine, Tetracaine, Dimethocaine, Benzocaine, Orthocaine, and Risocaine, or is not so modified
SKPSYXXTXK

SEQ ID NO: 69 - protein, 10 aa, Artificial Sequence
  Peptide component
  SITE 6,7: Wherein Xaa is hydroxyproline
  SITE 10: Wherein Lys is optionally C-terminally modified with a local anaesthetic selected from Procaine, Tetracaine, Dimethocaine, Benzocaine, Orthocaine, and Risocaine, or is not so modified
SKPSYXXTYK

SEQ ID NO: 70 - protein, 10 aa, Artificial Sequence
  Peptide component
  SITE 6,9: Wherein Xaa is hydroxyproline
  SITE 8: Wherein Xaa is 3,4-dihydroxyphenylalanine
  SITE 10: Wherein Lys is optionally C-terminally modified with a local anaesthetic selected from Procaine, Tetracaine, Dimethocaine, Benzocaine, Orthocaine, and Risocaine, or is not so modified
SKPSYXTXXK

SEQ ID NO: 71 - protein, 10 aa, Artificial Sequence
  Peptide component
  SITE 6,9: Wherein Xaa is hydroxyproline
  SITE 10: Wherein Lys is optionally C-terminally modified with a local anaesthetic selected from Procaine, Tetracaine, Dimethocaine, Benzocaine, Orthocaine, and Risocaine, or is not so modified
SKPSYXTYXK

SEQ ID NO: 72 - protein, 11 aa, Artificial Sequence
  Peptide component
  SITE 6,7,10: Wherein Xaa is hydroxyproline
  SITE 9: Wherein Xaa is 3,4-dihydroxyphenylalanine
  SITE 10: Wherein Lys is optionally C-terminally modified with a local anaesthetic selected from Procaine, Tetracaine, Dimethocaine, Benzocaine, Orthocaine, and Risocaine, or is not so modified
AKPSYXXTXXK

SEQ ID NO: 73 - protein, 11 aa, Artificial Sequence
  Peptide component
  SITE 6,7,10: Wherein Xaa is hydroxyproline
  SITE 11: Wherein Lys is optionally C-terminally modified with a local anaesthetic selected from Procaine, Tetracaine, Dimethocaine, Benzocaine, Orthocaine, and Risocaine, or is not so modified
AKPSYXXTYXK

SEQ ID NO: 74 - protein, 10 aa, Artificial Sequence
  Peptide component
  SITE 6,9: Wherein Xaa is hydroxyproline
  SITE 8: Wherein Xaa is 3,4-dihydroxyphenylalanine
AKPSYXTXXK

SEQ ID NO: 75 - protein, 10 aa, Artificial Sequence
  Peptide component
  SITE 6,9: Wherein Xaa is hydroxyproline
AKPSYXTYXK

SEQ ID NO: 76 - protein, 9 aa, Artificial Sequence
  Peptide component
  SITE 5,6,9: Wherein Xaa is hydroxyproline
  SITE 8: Wherein Xaa is 3,4-dihydroxyphenylalanine
KPSYXXTXX

SEQ ID NO: 77 - protein, 9 aa, Artificial Sequence
  Peptide component
  SITE 5,6,9: Wherein Xaa is hydroxyproline
KPSYXXTYX

SEQ ID NO: 78 - protein, 8 aa, Artificial Sequence
  Peptide component
  SITE 5,8: Wherein Xaa is hydroxyproline
  SITE 7: Wherein Xaa is 3,4-dihydroxyphenylalanine
KPSYXTXX

SEQ ID NO: 79 - protein, 8 aa, Artificial Sequence
  Peptide component
  SITE 5,8: Wherein Xaa is hydroxyproline
KPSYXTYX

SEQ ID NO: 80 - protein, 9 aa, Artificial Sequence
  Peptide component
  SITE 6,7: Wherein Xaa is hydroxyproline
SKPSYXXTY

SEQ ID NO: 81 - protein, 9 aa, Artificial Sequence
  Peptide component
  SITE 6,9: Wherein Xaa is hydroxyproline
  SITE 8: Wherein Xaa is 3,4-dihydroxyphenylalanine
SKPSYXTXX

SEQ ID NO: 82 - protein, 9 aa, Artificial Sequence
  Peptide component
  SITE 6,9: Wherein Xaa is hydroxyproline
SKPSYXTYX

SEQ ID NO: 83 - protein, 10 aa, Artificial Sequence
  Peptide component
  SITE 6,7,10: Wherein Xaa is hydroxyproline
  SITE 9: Wherein Xaa is 3,4-dihydroxyphenylalanine
AKPSYXXTXX

SEQ ID NO: 84 - protein, 10 aa, Artificial Sequence
  Peptide component
  SITE 6,7,10: Wherein Xaa is hydroxyproline
AKPSYXXTYX

SEQ ID NO: 85 - protein, 9 aa, Artificial Sequence
  Peptide component
  SITE 6,9: Wherein Xaa is hydroxyproline
  SITE 8: Wherein Xaa is 3,4-dihydroxyphenylalanine
AKPSYXTXX

SEQ ID NO: 86 - protein, 9 aa, Artificial Sequence
  Peptide component
  SITE 6,9: Wherein Xaa is hydroxyproline
AKPSYXTYX

SEQ ID NO: 87 - protein, 9 aa, Artificial Sequence
  Peptide component
  SITE 6,7: Wherein Xaa is hydroxyproline
  SITE 9: Wherein Xaa is 3,4-dihydroxyphenylalanine
SKPSYXXTX

SEQ ID NO: 88 - protein, 10 aa, Artificial Sequence
  SITE 1: Wherein Ala is N-terminally modified with a 3,4-Dihydrocinnamic acid group
  Peptide component
  SITE 6,7: Wherein Xaa is hydroxyproline
AKPSYXXTYK

SEQ ID NO: 89 - protein, 10 aa, Artificial Sequence
  SITE 1: Wherein Ala is N-terminally modified with a 3,4-Dihydrocinnamic acid group
  Peptide component
  SITE 6,9: Wherein Xaa is hydroxyproline
AKPSYXTYXK

SEQ ID NO: 90 - protein, 10 aa, Artificial Sequence
  Peptide component
  SITE 5,8: Wherein Xaa is 3,4-dihydroxyphenylalanine
  SITE 6,9: Wherein Xaa is hydroxyproline
AKPSXXTXXK

SEQ ID NO: 91 - protein, 9 aa, Artificial Sequence
  Peptide component
AKPSPTYPK

SEQ ID NO: 92 - protein, 10 aa, Artificial Sequence
  Peptide component
  SITE 3,6,9: Wherein Xaa is hydroxyproline
AKXSYXTYXK

SEQ ID NO: 93 - protein, 10 aa, Artificial Sequence
  Peptide component
  SITE 3,6,9: Wherein Xaa is hydroxyproline
  SITE 5,8: Wherein Xaa is 3,4-dihydroxyphenylalanine
AKXSXXTXXK

SEQ ID NO: 94 - protein, 10 aa, Artificial Sequence
  Peptide component
  SITE 3,6,9: Wherein Xaa is hydroxyproline
  SITE 8: Wherein Xaa is 3,4-dihydroxyphenylalanine
AKXSYXTXXK

SEQ ID NO: 95 - protein, 9 aa, Artificial Sequence
  SITE 1: Wherein Ala is N-terminally modified with a 3,4-Dihydrocinnamic acid group
  Peptide component
  SITE 6,7: Wherein Xaa is hydroxyproline
AKPSYXXTY

SEQ ID NO: 96 - protein, 9 aa, Artificial Sequence
  SITE 1: Wherein Ala is N-terminally modified with a 3,4-Dihydrocinnamic acid group
  Peptide component
  SITE 6,9: Wherein Xaa is hydroxyproline
AKPSYXTYX

SEQ ID NO: 97 - protein, 9 aa, Artificial Sequence
  Peptide component
  SITE 5,8: Wherein Xaa is 3,4-dihydroxyphenylalanine
  SITE 6,9: Wherein Xaa is hydroxyproline
AKPSXXTXX

SEQ ID NO: 98 - protein, 8 aa, Artificial Sequence
  Peptide component
AKPSPTYP

SEQ ID NO: 99 - protein, 9 aa, Artificial Sequence
  Peptide component
  SITE 3,6,9: Wherein Xaa is hydroxyproline
AKXSYXTYX

SEQ ID NO: 100 - protein, 9 aa, Artificial Sequence
  Peptide component
  SITE 3,6,9: Wherein Xaa is hydroxyproline
  SITE 5,8: Wherein Xaa is 3,4-dihydroxyphenylalanine
AKXSXXTXX

SEQ ID NO: 101 - protein, 9 aa, Artificial Sequence
  Peptide component
  SITE 3,6,9: Wherein Xaa is hydroxyproline
  SITE 8: Wherein Xaa is 3,4-dihydroxyphenylalanine
AKXSYXTXX

SEQ ID NO: 102 - protein, 10 aa, Artificial Sequence
  Peptide component
  SITE 5: Wherein Xaa is 3,4-dihydroxyphenylalanine
  SITE 6,10: Wherein Xaa is hydroxyproline
AKPSXXTYKX

SEQ ID NO: 103 - protein, 10 aa, Artificial Sequence
  Peptide component
  SITE 3,6,10: Wherein Xaa is hydroxyproline
  SITE 5: Wherein Xaa is 3,4-dihydroxyphenylalanine
AKXSXXTYKX

SEQ ID NO: 104 - protein, 10 aa, Artificial Sequence
  Peptide component
  SITE 6,10: Wherein Xaa is hydroxyproline
AKPSYXTYKX

SEQ ID NO: 105 - protein, 10 aa, Artificial Sequence
  Peptide component
  SITE 3,6,10: Wherein Xaa is hydroxyproline
AKXSYXTYKX

SEQ ID NO: 106 - protein, 10 aa, Artificial Sequence
  Peptide component
  SITE 6,10: Wherein Xaa is hydroxyproline
  SITE 8: Wherein Xaa is 3,4-dihydroxyphenylalanine
AKPSYXTXKX

SEQ ID NO: 107 - protein, 10 aa, Artificial Sequence
  Peptide component
  SITE 3,6,10: Wherein Xaa is hydroxyproline
  SITE 8: Wherein Xaa is 3,4-dihydroxyphenylalanine
AKXSYXTXKX

SEQ ID NO: 108 - protein, 10 aa, Artificial Sequence
  Peptide component
  SITE 5,8: Wherein Xaa is 3,4-dihydroxyphenylalanine
  SITE 6,10: Wherein Xaa is hydroxyproline
AKPSXXTXKX

SEQ ID NO: 109 - protein, 10 aa, Artificial Sequence
  Peptide component
  SITE 3,6,10: Wherein Xaa is hydroxyproline
  SITE 5,8: Wherein Xaa is 3,4-dihydroxyphenylalanine
AKXSXXTXKX

SEQ ID NO: 110 - protein, 7 aa, Artificial Sequence
  Peptide component
  VARIANT 2: Wherein Xaa is selected from proline, hydroxyproline, or dihydroxyproline
  VARIANT 4: Wherein Xaa is absent or is selected from tyrosine or 3,4-dihydroxyphenylalanine
  VARIANT 5: Wherein Xaa is selected from serine, proline, hydroxyproline, or dihydroxyproline
  VARIANT 6: Wherein Xaa is selected from proline, hydroxyproline, dihydroxyproline, threonine, 3,4-dihydroxyphenylalanine, or tyrosine
KXSXXXK

SEQ ID NO: 111 - protein, 7 aa, Artificial Sequence
  Peptide component
  SITE 5: Wherein Xaa is hydroxyproline
  SITE 6: Wherein Xaa is 3,4-dihydroxyphenylalanine
KPSYXXK

SEQ ID NO: 112 - protein, 7 aa, Artificial Sequence
  Peptide component
  SITE 5: Wherein Xaa is hydroxyproline
KPSYXYK

SEQ ID NO: 113 - protein, 4 aa, Artificial Sequence
  Amino acid sequence defined by Y
  VARIANT 2,3: Wherein Xaa is selected from proline, alanine, hydroxyproline, threonine, 3,4-dihydroxyphenylalanine, or tyrosine
PXXK

SEQ ID NO: 114 - protein, 4 aa, Artificial Sequence
  SITE 1: Wherein Xaa is hydroxyproline
  Amino acid sequence defined by Y
  VARIANT 2,3: Wherein Xaa is selected from proline, alanine, hydroxyproline, threonine, 3,4-dihydroxyphenylalanine, or tyrosine
XXXK

SEQ ID NO: 115 - protein, 4 aa, Artificial Sequence
  Amino acid sequence defined by Y
  VARIANT 2,3: Wherein Xaa is selected from proline, alanine, hydroxyproline, threonine, 3,4-dihydroxyphenylalanine, or tyrosine
TXXK

SEQ ID NO: 116 - protein, 4 aa, Artificial Sequence
  Amino acid sequence defined by Y
  SITE 3: Wherein Xaa is 3,4-dihydroxyphenylalanine
PTXK

SEQ ID NO: 117 - protein, 4 aa, Artificial Sequence
  Amino acid sequence defined by Y
PTYK

SEQ ID NO: 118 - protein, 4 aa, Artificial Sequence
  Amino acid sequence defined by Y
TYPK

SEQ ID NO: 119 - protein, 4 aa, Artificial Sequence
  Amino acid sequence defined by Y
  SITE 2: Wherein Xaa is 3,4-dihydroxyphenylalanine
TXPK

SEQ ID NO: 120 - protein, 4 aa, Artificial Sequence
  SITE 1: Wherein Xaa is hydroxyproline
  Amino acid sequence defined by Y
XTYK

SEQ ID NO: 121 - protein, 4 aa, Artificial Sequence
  SITE 1: Wherein Xaa is hydroxyproline
  Amino acid sequence defined by Y
  SITE 3: Wherein Xaa is 3,4-dihydroxyphenylalanine
XTXK

SEQ ID NO: 122 - protein, 4 aa, Artificial Sequence
  SITE 1: Wherein Xaa is hydroxyproline
  Amino acid sequence defined by Y
XTAK

SEQ ID NO: 123 - protein, 4 aa, Artificial Sequence
  Amino acid sequence defined by Y
  SITE 3: Wherein Xaa is hydroxyproline
TYXK

SEQ ID NO: 124 - protein, 4 aa, Artificial Sequence
  Amino acid sequence defined by Y
  SITE 2: Wherein Xaa is 3,4-dihydroxyphenylalanine
  SITE 3: Wherein Xaa is hydroxyproline
TXXK

SEQ ID NO: 125 - protein, 4 aa, Artificial Sequence
  Amino acid sequence defined by Y
  SITE 3: Wherein Xaa is hydroxyproline
TAXK

SEQ ID NO: 126 - protein, 5 aa, Artificial Sequence
  SITE 1,4: Wherein Xaa is hydroxyproline
  Amino acid sequence defined by Y
XTYXK

SEQ ID NO: 127 - protein, 5 aa, Artificial Sequence
  Amino acid sequence defined by Y
  SITE 3: Wherein Xaa is hydroxyproline
  SITE 5: Wherein Xaa is 3,4-dihydroxyphenylalanine
TYXKX

SEQ ID NO: 128 - protein, 5 aa, Artificial Sequence
  SITE 1,4: Wherein Xaa is hydroxyproline
  Amino acid sequence defined by Y
  SITE 3: Wherein Xaa is 3,4-dihydroxyphenylalanine
XTXXK